SEQUENCE LISTING (84 sequences)

SEQ ID NO: 1 - DNA, 544 bp, Artificial Sequence
  Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 2 - DNA, 335 bp, Artificial Sequence
  Synthetic polynucleotide
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtctcgaggccgccccgactgcatctgcgtgttccaattcgccaatgacaagacgctgggcggggtttgtgtcatcatagaactaaagacatgcaaatatatttcttccggggacaccgccagcaaacgcgagcaacgggccacggggatgaagcag

SEQ ID NO: 3 - DNA, 180 bp, Artificial Sequence
  Synthetic polynucleotide
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgt

SEQ ID NO: 4 - DNA, 5 bp, Artificial Sequence
  Synthetic poly nucleotide
ggaaa

SEQ ID NO: 5 - DNA, 15 bp, Artificial Sequence
  Synthetic polynucleotide
ggaaaaactgtttca

SEQ ID NO: 6 - DNA, 10 bp, Artificial Sequence
  Synthetic polynucleotide
gggactttcc

SEQ ID NO: 7 - DNA, 12 bp, Artificial Sequence
  Synthetic polynucleotide
  misc_feature (7)..(8): n is a, c, g, or t
agtttcnntttc

SEQ ID NO: 8 - DNA, 12 bp, Artificial Sequence
  Synthetic polynucleotide
agtttcacttct

SEQ ID NO: 9 - DNA, 12 bp, Artificial Sequence
  Synthetic polynucleotide
agtttcccgttc

SEQ ID NO: 10 - DNA, 12 bp, Artificial Sequence
  Synthetic poly nucleotide
agtttctctttc

SEQ ID NO: 11 - DNA, 12 bp, Artificial Sequence
  Synthetic polynucleotide
attctctcctcc

SEQ ID NO: 12 - DNA, 12 bp, Artificial Sequence
  Synthetic polynucleotide
agtttcttttct

SEQ ID NO: 13 - protein, 123 aa, Artificial Sequence
  Synthetic peptide
LLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 14 - DNA, 149 bp, Artificial Sequence
  Synthetic peptide
gccgccccgactgcatctgcgtgttccaattcgccaatgacaagacgctgggcggggtttgtgtcatcatagaactaaagacatgcaaatatatttcttccggggacaccgccagcaaacgcgagcaacgggccacggggatgaagcag

SEQ ID NO: 15 - protein, 8 aa, Artificial Sequence
  Synthetic peptide
GFTFSSYA

SEQ ID NO: 16 - protein, 8 aa, Artificial Sequence
  Synthetic peptide
IYSGGSST

SEQ ID NO: 17 - protein, 11 aa, Artificial Sequence
  Synthetic peptide
ARTSYLNHGDY

SEQ ID NO: 18 - protein, 8 aa, Artificial Sequence
  Synthetic peptide
RSNIGSDY

SEQ ID NO: 19 - protein, 3 aa, Artificial Sequence
  Synthetic peptide
GDN

SEQ ID NO: 20 - protein, 11 aa, Artificial Sequence
  Synthetic peptide
GTWDYTLNGVV

SEQ ID NO: 21 - protein, 118 aa, Artificial Sequence
  Synthetic peptide
QVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSS

SEQ ID NO: 22 - protein, 111 aa, Artificial Sequence
  Synthetic peptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLG

SEQ ID NO: 23 - protein, 250 aa, Artificial Sequence
  Synthetic peptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSS

SEQ ID NO: 24 - protein, 243 aa, Artificial Sequence
  Synthetic peptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 25 - protein, 7 aa, Artificial Sequence
  Synthetic peptide
TSGGGGS

SEQ ID NO: 26 - protein, 500 aa, Artificial Sequence
  Synthetic peptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 27 - protein, 21 aa, Artificial Sequence
  Synthetic peptide
SRGGGGSGGGGSGGGGSLEMA

SEQ ID NO: 28 - protein, 234 aa, Artificial Sequence
  Synthetic peptide
EVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSS

SEQ ID NO: 29 - protein, 111 aa, Artificial Sequence
  Synthetic peptide
QSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLG

SEQ ID NO: 30 - protein, 20 aa, Artificial Sequence
  Synthetic peptide
SETDTLLLWVLLLWVPGSTG

SEQ ID NO: 31 - protein, 249 aa, Artificial Sequence
  Synthetic peptide
QSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSS

SEQ ID NO: 32 - skipped

SEQ ID NO: 33 - protein, 39 aa, Artificial Sequence
  Synthetic peptide
IEVMYPPPYLDNEKSNGTIIHVKGKHLCPSPLFPGPSKP

SEQ ID NO: 34 - protein, 27 aa, Artificial Sequence
  Synthetic peptide
FWVLVVVGGVLACYSLLVTVAFIIFWV

SEQ ID NO: 35 - protein, 41 aa, Artificial Sequence
  Synthetic peptide
RSKRSRLLHSDYMNMTPRRPGPTRKHYQPYAPPRDFAAYRS

SEQ ID NO: 36 - protein, 112 aa, Artificial Sequence
  Synthetic peptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 120 aa, Artificial Sequence
  Synthetic peptide
QVQLVQSGAEVKKPGASVKLSCKASGYTFTYFDINWVRQTPEQGLEWMGGISPGDGNTNYNENFKGRVTMTRDTSSSTAYMELSRLRSDDTAVYYCARDGNFPYYAMDSWGQGTTVTVSS

SEQ ID NO: 38 - protein, 15 aa, Artificial Sequence
  Synthetic peptide
GGGGSGGGGSGGGGS

SEQ ID NO: 39 - protein, 113 aa, Artificial Sequence
  Synthetic peptide
DVVMTQSPLSLPVTLGEPASISCRSSQSLVYSNGNTYLHWYQQKPGQSPRLLIYKVSNRFSGVPDRFSGSGSGTDFTLKISRVEAEDVGVYFCSQSTHVPYTFGQGTKLEIKS

SEQ ID NO: 40 - protein, 5 aa, Artificial Sequence
  Synthetic peptide
GGGGS

SEQ ID NO: 41 - protein, 125 aa, Artificial Sequence
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 42 - protein, 15 aa, Artificial Sequence
  Synthetic peptide
GGGGSGGGGSGGGGS

SEQ ID NO: 43 - protein, 109 aa, Artificial Sequence
  Synthetic peptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 5 aa, Artificial Sequence
  Synthetic peptide
YFDIN

SEQ ID NO: 45 - protein, 12 aa, Artificial Sequence
  Synthetic peptide
GDGNTNYNENFK

SEQ ID NO: 46 - protein, 11 aa, Artificial Sequence
  Synthetic peptide
DGNFPYYAMDS

SEQ ID NO: 47 - protein, 16 aa, Artificial Sequence
  Synthetic peptide
RSSQSLVYSNGNTYLH

SEQ ID NO: 48 - protein, 7 aa, Artificial Sequence
  Synthetic peptide
KVSNRFS

SEQ ID NO: 49 - protein, 9 aa, Artificial Sequence
  Synthetic peptide
SQSTHVPYT

SEQ ID NO: 50 - protein, 5 aa, Artificial Sequence
  Synthetic peptide
KYAMN

SEQ ID NO: 51 - protein, 19 aa, Artificial Sequence
  Synthetic peptide
RIRSKYNNYATYYADSVKD

SEQ ID NO: 52 - protein, 14 aa, Artificial Sequence
  Synthetic peptide
HGNFGNSYISYWAY

SEQ ID NO: 53 - protein, 14 aa, Artificial Sequence
  Synthetic peptide
GSSTGAVTSGNYPN

SEQ ID NO: 54 - protein, 7 aa, Artificial Sequence
  Synthetic peptide
GTKFLAP

SEQ ID NO: 55 - protein, 9 aa, Artificial Sequence
  Synthetic peptide
VLWYSNRWV

SEQ ID NO: 56 - protein, 41 aa, Artificial Sequence; identical to SEQ ID NO: 35
  Synthetic peptide

SEQ ID NO: 57 - protein, 42 aa, Artificial Sequence
  Synthetic peptide
KRGRKKLLYIFKQPFMRPVQTTQEEDGCSCRFPEEEEGGCEL

SEQ ID NO: 58 - protein, 42 aa, Artificial Sequence
  Synthetic peptide
ALYLLRRDQRLPPDAHKPPGGGSFRTPIQEEQADAHSTLAKI

SEQ ID NO: 59 - protein, 248 aa, Artificial Sequence
  Synthetic peptide
DIVMTQTPLSLPVTPGEPASISCRSSQSLVHSNGNTYLHWYQQKPGKAPKLLIYKVSYRFSGVPDRFSGSGSGTDFTLKISRVEAEDVGVYYCSQNTHVPRTFGQGTKVEIKRSRGGGGSGGGGSGGGGSLEQVQLQESGPGLVKPSQTLSLTCTVSGYTFTNYYVFWVRQARGQRLEWIGDINPVNGDTNFNEKFKNRVTISADKSISTAYLQWSSLKASDTAMYYCARGGYTMDYWGQGTLVTVSS

SEQ ID NO: 60 - protein, 116 aa, Artificial Sequence
  Synthetic peptide
QVQLQESGPGLVKPSQTLSLTCTVSGYTFTNYYVFWVRQARGQRLEWIGDINPVNGDTNFNEKFKNRVTISADKSISTAYLQWSSLKASDTAMYYCARGGYTMDYWGQGTLVTVSS

SEQ ID NO: 61 - protein, 113 aa, Artificial Sequence
  Synthetic peptide
DIVMTQTPLSLPVTPGEPASISCRSSQSLVHSNGNTYLHWYQQKPGKAPKLLIYKVSYRFSGVPDRFSGSGSGTDFTLKISRVEAEDVGVYYCSQNTHVPRTFGQGTKVEIKR

SEQ ID NO: 62 - protein, 130 aa, Artificial Sequence
  Synthetic peptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSS

SEQ ID NO: 63 - protein, 109 aa, Artificial Sequence
  Synthetic peptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLG

SEQ ID NO: 64 - protein, 120 aa, Artificial Sequence
  Synthetic peptide
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 65 - protein, 106 aa, Artificial Sequence
  Synthetic peptide
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 66 - protein, 248 aa, Artificial Sequence
  Synthetic peptide
QVQLVQSGAEVKKPGASVKLSCKASGYTFTYFDINWVRQTPEQGLEWMGGISPGDGNTNYNENFKGRVTMTRDTSSSTAYMELSRLRSDDTAVYYCARDGNFPYYAMDSWGQGTTVTVSSGGGGSGGGGSGGGGSDVVMTQSPLSLPVTLGEPASISCRSSQSLVYSNGNTYLHWYQQKPGQSPRLLIYKVSNRFSGVPDRFSGSGSGTDFTLKISRVEAEDVGVYFCSQSTHVPYTFGQGTKLEIKS

SEQ ID NO: 67 - protein, 9 aa, Artificial Sequence
  Synthetic peptide
FMNKFIYEI

SEQ ID NO: 68 - protein, 580 aa, Artificial Sequence
  Synthetic peptide
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 69 - protein, 9 aa, Artificial Sequence
  Synthetic peptide
KLQCVDLHV

SEQ ID NO: 70 - DNA, 1473 bp, Artificial Sequence
  Synthetic polynucleotide
atggaaaccgatacactgctgctgtgggtgctgctgctgtgggtgccaggatccacaggccagagcgtgctgacacagcctgcctccgtgtctggctctcctggccagtccatcaccatcagctgtaccggcaccagctccgacgtgggcggctacaattacgtgtcctggtatcagcagcatcccggcaaggcccccaagctgatgatctacgacgtgaacaaccggcccagcgaggtgtccaacagattcagcggcagcaagagcggcaacaccgccagcctgacaatcagcggactgcaggccgaggacgaggccgactactactgcagcagctacaccaccggcagcagagccgtgtttggcggaggcaccaagctgacagtgctgggcagtagaggcggcggaggaagcggaggcggaggatctggcggagggggatctctggaaatggccgaggtgcagctggtgcagtctggcgccgaagtgaagaagcctggcgagagcctgaccatctcctgcaaggccagcggctacagcttccccaactactggatcacctgggtgcgccagatgtccggcggaggcctggaatggatgggcagaatcgaccccggcgacagctacacaacctacaaccccagcttccagggccacgtgaccatcagcatcgacaagagcaccaataccgcctacctgcactggaacagcctgaaggcctccgacaccgccatgtactactgcgcccggtactatgtgtccctggtggatatctggggccagggcacactcgtgaccgtgtctagcgccgctgcaattgaagtgatgtacccccctccctacctggacaacgagaagtccaacggcaccatcatccacgtgaagggcaagcacctgtgccccagccctctgtttcctgggccctccaagcctttctgggtgctggtggtcgtgggcggagtgctggcctgttactccctgctcgtgacagtggccttcatcatcttttgggtgcgcagcaagcggagcagactgctgcacagcgactacatgaacatgacccccagacggccaggccccaccagaaagcactaccagccttacgcccctcccagagacttcgccgcctaccggtccagagtgaagttcagcagaagcgccgacgcccctgcctaccagcagggacagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccacaaaggacacctacgacgcactgcacatgcaggccctgccccccaga

SEQ ID NO: 71 - DNA, 60 bp, Artificial Sequence
  Synthetic polynucleotide
atggaaaccgatacactgctgctgtgggtgctgctgctgtgggtgccaggatccacaggc

SEQ ID NO: 72 - DNA, 747 bp, Artificial Sequence
  Synthetic polynucleotide
cagagcgtgctgacacagcctgcctccgtgtctggctctcctggccagtccatcaccatcagctgtaccggcaccagctccgacgtgggcggctacaattacgtgtcctggtatcagcagcatcccggcaaggcccccaagctgatgatctacgacgtgaacaaccggcccagcgaggtgtccaacagattcagcggcagcaagagcggcaacaccgccagcctgacaatcagcggactgcaggccgaggacgaggccgactactactgcagcagctacaccaccggcagcagagccgtgtttggcggaggcaccaagctgacagtgctgggcagtagaggcggcggaggaagcggaggcggaggatctggcggagggggatctctggaaatggccgaggtgcagctggtgcagtctggcgccgaagtgaagaagcctggcgagagcctgaccatctcctgcaaggccagcggctacagcttccccaactactggatcacctgggtgcgccagatgtccggcggaggcctggaatggatgggcagaatcgaccccggcgacagctacacaacctacaaccccagcttccagggccacgtgaccatcagcatcgacaagagcaccaataccgcctacctgcactggaacagcctgaaggcctccgacaccgccatgtactactgcgcccggtactatgtgtccctggtggatatctggggccagggcacactcgtgaccgtgtctagc

SEQ ID NO: 73 - DNA, 117 bp, Artificial Sequence
  Synthetic polynucleotide
attgaagtgatgtacccccctccctacctggacaacgagaagtccaacggcaccatcatccacgtgaagggcaagcacctgtgccccagccctctgtttcctgggccctccaagcct

SEQ ID NO: 74 - DNA, 81 bp, Artificial Sequence
  Synthetic polynucleotide
ttctgggtgctggtggtcgtgggcggagtgctggcctgttactccctgctcgtgacagtggccttcatcatcttttgggtg

SEQ ID NO: 75 - DNA, 123 bp, Artificial Sequence
  Synthetic polynucleotide
cgcagcaagcggagcagactgctgcacagcgactacatgaacatgacccccagacggccaggccccaccagaaagcactaccagccttacgcccctcccagagacttcgccgcctaccggtcc

SEQ ID NO: 76 - DNA, 336 bp, Artificial Sequence
  Synthetic polynucleotide
agagtgaagttcagcagaagcgccgacgcccctgcctaccagcagggacagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccacaaaggacacctacgacgcactgcacatgcaggccctgccccccaga

SEQ ID NO: 77 - protein, 491 aa, Artificial Sequence
  Synthetic peptide
METDTLLLWVLLLWVPGSTGQSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 502 aa, Artificial Sequence
  Synthetic peptide
QVQLVQSGAEVKKPGASVKLSCKASGYTFTYFDINWVRQTPEQGLEWMGGISPGDGNTNYNENFKGRVTMTRDTSSSTAYMELSRLRSDDTAVYYCARDGNFPYYAMDSWGQGTTVTVSSGGGGSGGGGSGGGGSDVVMTQSPLSLPVTLGEPASISCRSSQSLVYSNGNTYLHWYQQKPGQSPRLLIYKVSNRFSGVPDRFSGSGSGTDFTLKISRVEAEDVGVYFCSQSTHVPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 79 - protein, 8 aa, Artificial Sequence
  Synthetic peptide
GYSFPNYW

SEQ ID NO: 80 - protein, 8 aa, Artificial Sequence
  Synthetic peptide
IDPGDSYT

SEQ ID NO: 81 - protein, 10 aa, Artificial Sequence
  Synthetic peptide
ARYYVSLVDI

SEQ ID NO: 82 - protein, 9 aa, Artificial Sequence
  Synthetic peptide
SSDVGGYNY

SEQ ID NO: 83 - protein, 3 aa, Artificial Sequence
  Synthetic peptide
DVN

SEQ ID NO: 84 - protein, 10 aa, Artificial Sequence
  Synthetic peptide
SSYTTGSRAV

(19) United States
(12) Patent Application Publication
(10) Pub. No.: US 2020/0255505 A1
(43) Pub. Date: Aug. 13, 2020

(54) Cells Expressing Cell Surface Receptors and Antibodies

(71) Applicant: EUREKA THERAPEUTICS, INC., Emeryville, CA (US)

(72) Inventors: Binnaz K. Staley, San Carlos, CA (US); Fei Xie, San Mateo, CA (US); Lucas Horan, Emeryville, CA (US); Yiyang Xu, Pleasanton, CA (US); Javier Morales, San Jose, CA (US); Hong Liu, El Sobrante, CA (US)

(21) Appl. No.: 16/637,524

(22) PCT Filed: Aug. 8, 2018
(86) PCT No.: PCT/US18/45805

§ 371 Date: Feb. 7, 2020

Related U.S. Application Data
(60) Provisional application No. 62/543,102, filed on Aug. 9, 2017.

(51) Int. Cl. C07K 16/18; C07K 16/28; C07K 14/705; C07K 14/725; C12N 15/11; C12N 5/0783; C12N 15/86; A61K 35/17; A61P 35/00

(52) U.S. Cl. CPC C07K 16/18; C12N 2740/15043; C07K 14/70521; C07K 14/7051; C07K 14/70575; C12N 15/11; C12N 5/0636; C12N 15/86; A61K 35/17; A61P 35/00; C07K 2317/622; C07K 2317/565; C07K 2319/03; C07K 2319/33; C12N 2510/00; C07K 16/2863

(57) ABSTRACT

The present disclosure provides a cell comprising (a) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. Related compositions, kits, articles of manufacture, and methods are further provided.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 66,085 bytes ACII (Text) file named “51927_SeqListing.txt,” created on Aug. 8, 2017.

### BACKGROUND

A different approach has been developed in recent years to engage T cells for targeted cancer immunotherapy. This new approach is called Chimeric Antigen Receptor T cell Therapy (CAR-T). It merges the exquisite targeting specificity of monoclonal antibodies with the potent cytotoxicity and long-term persistence provided by cytotoxic T cells. A CAR is composed of an extracellular domain that recognizes a cell surface antigen, a transmembrane region, and an intracellular signaling domain. The extracellular domain consists of the antigen-binding variable regions from the heavy and light chains of a monoclonal antibody that are fused into a single-chain variable fragment (scFv). The intracellular signaling domain contains an immunoreceptor tyrosine-based activation motif (ITAM), such as those from CD3ζ or FcRγ, and one or more costimulatory signaling domains, such as those from CD28, 4-1BB or OX40 (Barrett D M et al., Annu. Rev. Med. 65:333-47, 2014; Davila M L et al., Oncoimmunology. 1(9):1577-1583, 2012). Binding of target antigens by CARs grafted onto a T cell surface can trigger T cell effector functions independent of TCR-peptide/MHC complex interaction. Thus, T cells equipped with CARs can be redirected to attack a broad variety of cells, including those that do not match the MHC type of the TCRs on the T cells but express the target cell-surface antigens. This approach overcomes the constraints of MHC-restricted TCR recognition and avoids tumor escape through impairments in antigen presentation or MHC molecule expression. Clinical trials have shown clinically significant antitumor activity of CAR-T therapy in neuroblastoma (Louis C U et al., Blood. 118(23):6050-6056, 2011), B-ALL (Maude, S L, et al., New England Journal of Medicine 371:16:1507-1517, 2014), CLL (Brentjens, R J, et al. Blood 118:18:4817-4828, 2011), and B cell lymphoma (Kochenderfer, J N, et al. Blood 116:20:4099-4102, 2010). In one study, a 90% complete remission rate in 30 patients with B-ALL treated with CD19-CAR T therapy was reported (Maude, S L, et al., supra).

Most if not all CARs studied so far have been directed to tumor antigens with high cell surface expression. To target low-copy number cell-surface tumor antigens and intracellular tumor antigens, which represent 95% of all known tumor-specific antigens, there is a need to develop more potent and effective engineered cell therapies (Cheever, et al., Clin. Cancer Res. 15(17):5323-37, 2009).

The success achieved with CAR-T cells in B cell cancers has prompted researchers to evaluate such engineered cells as a therapeutic for other diseases, including solid tumors. In fact, clinical trials have already been carried out with CAR-T cells that recognize solid tumor antigens, including, for example, human epidermal growth factor receptor-2 (HER-2), carcinoembryonic antigen (CEA), and prostate-specific membrane antigen (PSMA), though the preliminary results of such trials have been discouraging.

Additional studies with CAR-T cells have highlighted the need for enhancing the safety and selectivity of CAR-T cells. The potential for off-target/off-tumor toxicity remains a limitation to this therapeutic strategy. Other issues, such as antigen escape, pose other challenges to the effectiveness of CAR-T cells.

Thus, there is a need in the art for improved engineered cells, e.g., improved CAR-T cells, with enhanced safety and selectivity.

### SUMMARY

The present disclosure provides a cell comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, the expression of the second nucleotide sequence is activated only when the first target binds to the cell surface receptor.

The present disclosure provides also provides a T-cell comprising (a) a first nucleic acid comprising a first nucleotide sequence encoding a chimeric antigen receptor (CAR) comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding a bispecific antibody which binds to a second target and a third target, wherein the second nucleotide sequence is operably linked to an inducible promoter, wherein expression of the second nucleotide sequence is activated upon binding of the first target to the CAR, wherein each of the first target and third target is an antigen expressed by a solid tumor, wherein the first target is different from the third target, and wherein the second target is expressed on the surface of a T-cell or a natural killer cell.

Further provided is a T-cell comprising: (a) a first nucleic acid comprising a first nucleotide sequence encoding a chimeric antigen receptor (CAR) comprising an extracellular domain (ECD) which binds to an alpha fetoprotein (AFP) peptide bound to an MHC molecule, a transmembrane domain (TMD), and an intracellular domain (ICD) comprising at least a portion of CD28 and CD3, and (b) a second nucleic acid comprising a second nucleotide sequence encoding a bispecific antibody which binds to GPC3 and CD3, wherein the second nucleotide sequence is operably linked to an inducible NFAT promoter, wherein expression of the second nucleotide sequence is activated upon binding of the AFP peptide bound to the MHC molecule to the CAR.

The present disclosure also provides a composition comprising (i) a nucleic acid comprising a first nucleotide sequence encoding a cell surface receptor comprising an ECD which binds to a first target, a TMD, and an ICD comprising at least a portion of a T-cell signaling molecule, (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, the expression of the second nucleotide sequence is activated only when the first target binds to the cell surface receptor.

The present disclosure furthermore provides a method of making a therapeutic cell. In exemplary embodiments, the method comprises contacting a cell with the composition of the present disclosure, e.g., the composition comprising (i) a nucleic acid comprising a first nucleotide sequence encoding a cell surface receptor comprising an ECD which binds to a first target, a TMD, and an ICD comprising at least a portion of a T-cell signaling molecule, (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, the expression of the second nucleotide sequence is activated only when the first target binds to the cell surface receptor.

Pharmaceutical compositions are provided by the present disclosure. In exemplary embodiments, the pharmaceutical composition comprises cells comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor, and a pharmaceutically acceptable carrier, diluent, or excipient. In exemplary aspects, the expression of the second nucleotide sequence is activated only when the first target binds to the cell surface receptor. In exemplary embodiments, the pharmaceutical composition comprises cells produced by the method of making a therapeutic cell described herein, and a pharmaceutically acceptable carrier, diluent, or excipient. In exemplary embodiments, the pharmaceutical composition comprises the composition of the present disclosure, e.g., comprising a nucleic acid comprising a first nucleotide sequence and a nucleic acid comprising a second nucleotide sequence, and a pharmaceutically acceptable carrier, diluent, or excipient.

Articles of manufacture are also provided by the present disclosure. In exemplary embodiments, the article of manufacture comprises cells comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor, housed in a container. In exemplary aspects, the expression of the second nucleotide sequence is activated only when the first target binds to the cell surface receptor. In exemplary embodiments, the article of manufacture comprises cells produced by the method of making a therapeutic cell described herein, and is housed in a container. In exemplary embodiments, the article of manufacture comprises the composition of the present disclosure, e.g., comprising a nucleic acid comprising a first nucleotide sequence and a nucleic acid comprising a second nucleotide sequence, housed in a container.

Further provided are kits. In exemplary embodiments, the kit comprises cells comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor, and a device for administration of the cells. In exemplary aspects, the expression of the second nucleotide sequence is activated only when the first target binds to the cell surface receptor. In exemplary embodiments, the kit comprises cells produced by the method of making a therapeutic cell described herein, and a device for administration of the therapeutic cells. In exemplary embodiments, the kit comprises the composition of the present disclosure, e.g., comprising a nucleic acid comprising a first nucleotide sequence and a nucleic acid comprising a second nucleotide sequence, and a device for administration of the composition.

The present disclosure provides a method of killing a diseased or infected cell. In exemplary embodiments, the method comprises contacting the diseased or infected cell with the cells of the present disclosure, e.g., comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, the expression of the second nucleotide sequence is activated only when the first target binds to the cell surface receptor.

A method of treating a subject with a disease is also provided by the present disclosure. In exemplary embodiments, the method comprises administering to the subject a pharmaceutical composition of the present disclosure in an amount effective to treat the disease, wherein the first target and third target are expressed by a cell of the disease.

The present disclosure also provides a method of treating a viral infection in a subject. In exemplary embodiments, the method comprises administering to the subject a pharmaceutical composition of the present disclosure in an amount effective to treat the viral infection, wherein the first target and/or third target are viral antigens.

The present disclosure further provides a method of treating a subject with a tumor or cancer or a viral infection, comprising (a) obtaining immune cells from a subject, (b) contacting the cells with a composition of the present disclosure, e.g., a composition comprising (i) a nucleic acid comprising a first nucleotide sequence encoding a cell surface receptor comprising an ECD which binds to a first target, a TMD, and an ICD comprising at least a portion of a T-cell signaling molecule, (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor, and (c) administering the cells to the subject in an amount effective to treat the tumor or cancer or viral infection. In exemplary aspects, the expression of the second nucleotide sequence is activated only when the first target binds to the cell surface receptor.

### BRIEF DESCRIPTION OF THE DRAWINGS

FIG. 1 is a set of graphs of the % specific lysis of HepG2 cells, HepG2-GPC3 knock out (ko) cells, SK-Hep1 cells, or SK-Hep1-GPC3 cells, by cells expressing a chimeric antigen receptor (CAR)-specific for alpha fetoprotein (AFP) (white bars) or an AFP-specific CAR in combination with a bispecific antibody specific for GPC3 and CD3 (black bars).

FIG. 2 is a graph of GMCSF or IFN-γ released by cells expressing a chimeric antigen receptor (CAR)-specific for alpha fetoprotein (AFP) (white bars) or an AFP-specific CAR in combination with a bispecific antibody (BsAb) specific for GPC3 and CD3 (black bars) stimulated by HepG2 cells or HepG2-GPC3 knock out (ko) cells.

FIG. 3 is a graph of IL-2 or TNF-α released by cells expressing a CAR-specific for AFP (white bars) or an AFP-specific CAR in combination with a BsAb specific for GPC3 and CD3 (black bars) stimulated by HepG2 cells or HepG2-GPC3 knock out (ko) cells.

FIG. 4A is an illustration of the structure of an anti-GPC3/Anti-CD3 BsAb.

FIG. 4B is an illustration of the structure of an anti-AFP CAR.

FIG. 4C is an illustration of the structure of an anti-PSMA/Anti-CD3 BsAb.

FIG. 5 is a vector map of the lentiviral vector encoding the anti-GPC3/anti-CD3 BsAb and the anti-AFP chimeric antigen receptor (CAR).

### DETAILED DESCRIPTION

The present disclosure provides a cell comprising (a) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, the expression of the second nucleotide sequence is activated only when the first target binds to the cell surface receptor.

Cell Type

In exemplary embodiments, the cell of the present disclosure is an immune cell or a cell of the immune system. Accordingly, the cell may be a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In exemplary aspects, the cell is a T lymphocyte. In exemplary aspects, the T lymphocyte is CD8+, CD4+, CD8+/CD4+, or a T-regulatory (T-reg) cell. In exemplary embodiments, the T lymphocyte is genetically engineered to silence the expression of an endogenous TCR. In exemplary aspects, the cell is a natural killer (NK) cell.

In exemplary embodiments, the cell of the present disclosure is a mammalian cell. As used herein, the term “mammal” refers to any vertebrate animal of the mammalia class, including, but not limited to, any of the monotreme, marsupial, and placental taxas. In some embodiments, the mammal is one of the mammals of the order Rodentia, such as mice and hamsters, and mammals of the order Logomorpha, such as rabbits. In exemplary embodiments, the mammals are from the order Carnivora, including Felines (cats) and Canines (dogs). In exemplary embodiments, the mammals are from the order Artiodactyla, including Bovines (cows) and S wines (pigs) or of the order Perssodactyla, including Equines (horses). In some instances, the mammals are of the order Primates, Ceboids, or Simoids (monkeys) or of the order Anthropoids (humans and apes). In particular embodiments, the mammal is a human.

In exemplary embodiments, the cell is a human immune cell, e.g., a human T-lymphocyte or human NK cell. In exemplary aspects, the cell is a cell isolated or obtained from a human. In exemplary aspects, the cell of the present disclosure is a lymphocyte isolated from human whole blood. In exemplary instances, the cell is a T-lymphocyte isolated from a fraction of PBMCs from human peripheral blood. In exemplary instances, the cell is a leukocyte separated from blood via leukapheresis.

Nucleic Acids

The cells and compositions of the present disclosure comprise a nucleic acid comprising a first nucleotide sequence and a nucleic acid comprising a second nucleotide sequence. In exemplary aspects, the nucleic acid comprising the first nucleotide sequence also comprises the second nucleotide sequence. In exemplary aspects, the nucleic acid comprises the first nucleotide sequence which is upstream of the second nucleotide sequence. In alternative aspects, the nucleic acid comprises the first nucleotide sequence which is downstream of the second nucleotide sequence. Alternatively, the nucleic acid comprising the first nucleotide sequence does not comprise the second nucleotide sequence, and the cells and compositions of the present disclosure comprise a first nucleic acid comprising a first nucleotide sequence and a second nucleic acid comprising a second nucleotide sequence, wherein the first nucleic acid is separate and distinct from the second nucleic acid.

In exemplary aspects, the nucleic acid is inserted into the genome of the cell. In exemplary aspects, the nucleic acid is not inserted into the genome of the cell. In exemplary aspects, the nucleic acid is a part of a vector. Suitable vectors are known in the art and described herein.

By “nucleic acid” as used herein includes “polynucleotide,” “oligonucleotide,” and “nucleic acid molecule,” and generally means a polymer of DNA or RNA or both DNA and RNA, or modified forms thereof. In exemplary aspects, the nucleic acid comprising the first and/or second nucleotide sequence is single-stranded or is double-stranded or comprises portions that are single-stranded and portions that are double-stranded. In exemplary aspects, the nucleic acid comprising the first and/or second nucleotide sequence is synthesized in whole or in part. In exemplary aspects, the nucleic acid comprising the first and/or second nucleotide sequence is partly obtained (e.g., isolated and/or purified) from a natural source. In exemplary aspects, the nucleic acid comprising the first and/or second nucleotide sequence comprises natural, non-natural and/or altered nucleotides. In exemplary aspects, the nucleic acid comprising the first and/or second nucleotide sequence comprises a natural, non-natural and/or altered inter-nucleotide linkage, such as a phosphoroamidate linkage or a phosphorothioate linkage, instead of the phosphodiester found between the nucleotides of an unmodified oligonucleotide.

In exemplary aspects, the nucleic acid comprises at least one non-naturally-occurring nucleotide. In exemplary aspects, the nucleic acid comprises a replacement of the hydroxyl at the 2′-position of ribose with an O-alkyl group, e.g., —O—CH₃, —OCH₂CH₃. In exemplary aspects, the nucleic acid comprises a modified ribonucleotide wherein the 2′ hydroxyl of ribose is modified to methoxy (OMe) or methoxy-ethyl (MOE) group. In exemplary aspects, the nucleic acid comprises a modified ribonucleotide wherein the 2′ hydroxyl of ribose is replaced with allyl, amino, azido, halo, thio, O-allyl, O—C₁-C₁₀ alkyl, O—C₁-C₁₀ substituted alkyl, O—C₁-C₁₀ alkoxy, O—C₁-C₁₀ substituted alkoxy, OCF₃, O(CH₂)₂SCH₃, O(CH₂)₂—O—N(R¹)(R²), or O(CH₂)—C(═O)—N(R¹)(R²), wherein each of R¹ and R² is independently selected from the group consisting of H, an amino protecting group or substituted or unsubstituted C₁-C₁₀ alkyl. In exemplary aspects, the nucleic acid comprises a modified ribonucleotide wherein the 2′ hydroxyl of ribose is replaced with 2′F, SH, CN, OCN, CF₃, O-alkyl, S-Alkyl, N(R¹)alkyl, O-alkenyl, S-alkenyl, or N(R¹)-alkenyl, O-alkynyl, S-alkynyl, N(R¹)-alkynyl, O-alkylenyl, O-Alkyl, alknyyl, alkaryl, aralkyl, O-alkaryl, or O-aralkyl. In exemplary aspects, the nucleic acid comprises a replacement of the hydrogen at the 2′-position of ribose with halo, e.g., F. In exemplary aspects, the nucleic acid comprises a fluorine derivative nucleic acid.

In exemplary aspects, the nucleic acid comprises a substituted ring. In exemplary aspects, the nucleic acid is or comprises a hexitol nucleic acid. In exemplary aspects, the nucleic acid is or comprises a nucleotide with a bicyclic or tricyclic sugar moiety. In exemplary aspects, the bicyclic sugar moiety comprises a bridge between the 4′ and 2′ furanose ring atoms.

In exemplary aspects, the nucleic acid comprises one or more modified nucleotides, including, but not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueuosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueuosine, 5′-methoxycarboxymethyluracil, 5-methoxyuracil, 2-methylthio-N⁶-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

In exemplary aspects, the nucleic acid comprises at least one non-naturally-occurring internucleotide linkage. In exemplary aspects, the nucleic acid comprises non-naturally-occurring nucleotides which differ from naturally occurring nucleotides by comprising a chemical group to replace the phosphate group. In exemplary aspects, the nucleic acid comprises or is a methylphosphonate oligonucleotide, which are noncharged oligomers in which a non-bridging oxygen atom, e.g., alpha oxygen of the phosphate, is replaced by a methyl group. In exemplary aspects, the nucleic acid comprises or is a phosphorothioate, wherein at least one of the non-bridging oxygen atom, e.g., alpha oxygen of the phosphate, is replaced by a sulfur. In exemplary aspects, the nucleic acid comprises or is a boranophosphate oligonucleotide, wherein at least one of the non-bridging oxygen atom, e.g., alpha oxygen of the phosphate, is replaced by —BH₃.

In exemplary aspects, the nucleic acid comprises a modified backbone. In exemplary aspects, the nucleic acid is or comprises a peptide nucleic acid (PNA) containing an uncharged flexible polyamide backbone comprising repeating N-(2-aminoethyl)glycine units to which the nucleobases are attached via methylene carbonyl linkers. In exemplary aspects, the nucleic acid comprises a backbone substitution. In exemplary aspects, the nucleic acid is or comprises an N3′→P5′ phosphoramidate, which results from the replacement of the oxygen at the 3′ position on ribose by an amine group. Such nucleic acid analogs are further described in Dias and Stein, Molec Cancer Ther 1: 347-355 (2002). In exemplary aspects, the nucleic acid comprises a nucleotide comprising a conformational lock. In exemplary aspects, the antisense nucleic acid analog is or comprises a locked nucleic acid.

In some aspects, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids in some aspects are constructed based on chemical synthesis and/or enzymatic ligation reactions using procedures known in the art. See, for example, Sambrook et al., supra; and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueuosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueuosine, 5′-methoxycarboxymethyluracil, 5-methoxyuracil, 2-methylthio-N⁶-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the present disclosure can be purchased from companies, such as Macromolecular Resources (Fort Collins, Colo.) and Synthegen (Houston, Tex.).

In exemplary aspects, the nucleic acid comprises regulatory sequences, such as transcription and translation initiation and termination codons, which are specific to the type of cell of the present disclosure. In exemplary aspects, the nucleic acid comprises regulatory sequences specific for transcription and translation by human polymerases, human ribosomes and/or human ribozymes.

Promoters

In exemplary embodiments, the nucleic acid comprising the first and/or second nucleotide sequence comprises a promoter and the nucleotide sequence(s) is/are operably linked to that promoter. In exemplary aspects, the promoter is a RNA polymerase II promoter. The promoter may be a strong promoter, a weak promoter, an inducible promoter, a tissue-specific promoter, or a combination thereof. In exemplary aspects, the promoter is a mammalian, yeast, or plant promoter. In exemplary aspects, the promoter is a viral promoter. In exemplary aspects, the promoter is a non-viral promoter or a viral promoter, e.g., a cytomegalovirus (CMV) promoter, an SV40 promoter, an Rous Sarcoma Virus (RSV) promoter, and a promoter found in the long-terminal repeat of the murine stem cell virus.

In exemplary aspects, the first and second nucleotide sequences are operably linked to the same promoter. In exemplary aspects, the promoter is located upstream of the first nucleotide sequence, which is, in turn, located upstream of the second nucleotide sequence. In alternative aspects, the promoter is located upstream of the second nucleotide sequence, which is, in turn, located upstream of the first nucleotide sequence.

In alternative aspects, the first nucleotide sequence is operably linked to a first promoter and the second nucleotide sequence is operably linked to a second promoter. In exemplary aspects, the first promoter is a constitutive promoter such that the first nucleotide sequence is operably linked to a constitutive promoter. Accordingly, in exemplary aspects, the first nucleotide sequence is constitutively expressed by the cell of the present disclosure.

Constitutive promoters are known in the art and include but are not limited to: cytomegalovirus (CMV) promoters, human elongation factor-1 alpha (EF-1α), Simian Virus 40 (SV40) promoters, Phosphoglycerate Kinase-1 (PGK1), Ubiquitin C (Ubc) promoters, human beta actin, CAG promoters, Actin C5 (Ac5) promoters, polyhedron, TEF1 promoters, GDS promoters, Cauliflower Mosaic Virus 35S (CaMV35S) promoters, Ubiquitin (Ubi) promoters, and the like. In exemplary aspects, the constitutive promoter is a human promoter. In exemplary aspects, the human promoter is human EF-1α) or a functional variant thereof. In exemplary aspects, the human promoter comprises a nucleotide sequence of SEQ ID NO: 1.

In exemplary aspects, the second promoter is an inducible promoter such that the second nucleotide sequence is operably linked to an inducible promoter. The term “inducible promoter” refers to a promoter whose activity can be regulated by adding or removing one or more specific signals. For example, an inducible promoter may activate transcription of an operably linked nucleic acid under a specific set of conditions, e.g., in the presence of an inducing agent or conditions that activates the promoter and/or relieves repression of the promoter. Accordingly, in exemplary aspects, the expression of the second nucleotide sequence by the cell of the present disclosure is inducible and occurs only under specific conditions. Inducible promoters are known in the art and include but are not limited to: Galactose-1 (GAL1) or Galactose-4 (GAL4) promoters, Calcium/calmodulin-dependent kinase II (CaMKIIa), Upstream Activating Sequence (UAS), TRE, and the like.

The cells of the present disclosure may be engineered such that expression of the second nucleotide sequence occurs only under a certain condition. In exemplary aspects, the condition under which the second nucleotide sequence is expressed is tied to the cell surface receptor encoded by the first nucleotide sequence. Without being bound to a particular theory, it is the selective and transient expression of the second nucleotide sequence that allows for the cells of the present disclosure to provide therapeutic potency with less off-target toxicity. The cells of the present disclosure advantageously provide the appropriate therapy at the right time and at the right place.

In exemplary instances, the expression of the second nucleotide sequence is activated only upon activation of the cell surface receptor encoded by the first nucleotide sequence. In exemplary aspects, the expression of the second nucleotide sequence is activated only upon binding of the first target to the cell surface receptor. In exemplary aspects, the inducible promoter comprises a binding site for a protein expressed or activated upon binding of the first target to the cell surface receptor. In exemplary aspects, the protein is a transcription factor expressed or activated in activated T-cells. Transcription factors expressed or activated in activated T-cells are known in the art and include, e.g., any one of the members of the Nuclear Factor of Activated T-Cells (NFAT) family of transcription factors, AP-1, and any one of the members of the NF-κB/Rel family of transcription factors. In exemplary instances, the member of the NFAT family of transcription factors is NFATc1 (NFATc, NFAT2); NFATc2 (NFATp, NFAT1); NFATc3 (NFATx, NFAT4); NFATc4 (NFAT3); or NFATc5 (TonEBP). In exemplary aspects, the member of the NF-κB/Rel family of transcription factors is p50, p65, RelB, c-Rel, and p52. In exemplary aspects, the transcription factor is one of IRF4, Rel, T-bet, Blimp-1, BATF, or Nur77. See, e.g., Conley et al., Frontiers in Immunology 7: 76 (2016); Badran et al., J Biol Chem 277: 47136-47148 (2002); and Latinis et al., J Biol Chem 272: 31427-31434 (1997). Accordingly, in some exemplary aspects, the inducible promoter comprises an NFAT consensus binding site comprising the sequence of 5′-GGAAA-3′ (SEQ ID NO: 4), an NFAT/AP-1 consensus binding site comprising the sequence of GGAAAAACTGTTTCA (SEQ ID NO: 5), and/or an NF-κB consensus binding site comprising the sequence of 5′-GGGACTTTCC-3′ (SEQ ID NO: 6). See, e.g., Kinoshita et al., Immunity 6:235-244 (1997), and Chytil and Verdine, Curr. Opin. Struct. Biol 6:91-100 (1996). In exemplary aspects, the inducible promoter comprises an Interferon (IFN)-stimulated response element (ISRE) comprising the sequence of AGTTTCNNTTTC (SEQ ID NO: 7), e.g., AGTTTCACTTCT (SEQ ID NO: 8), AGTTTCCCGTTC (SEQ ID NO: 9), AGTTTCTCTTTC (SEQ ID NO: 10), ATTCTCTCCTCC (SEQ ID NO; 11), and AGTTTCTTTTCT (SEQ ID NO: 12).

In exemplary aspects, the cell surface receptor is a T-cell receptor (TCR) or a modified form thereof and expression of the second nucleotide sequence occurs only when the appropriate antigen binds to the TCR and leads to T-cell activation and T-cell signaling.

In exemplary aspects, the cell comprises a nucleic acid comprising a third nucleotide sequence encoding a protein which is exogenous to the cell, wherein the third nucleotide sequence is operably linked to an inducible promoter comprising a binding site for a transcription factor expressed in activated T-cells, wherein the expression of the exogenous protein activates expression of the second nucleotide sequence. In exemplary aspects, binding of the first target to the cell surface receptor encoded by the first nucleotide sequence causes expression or activation of a transcription factor expressed or activated in activated T-cells. The binding of the transcription factor leads to expression of the third nucleotide sequence, which, in turn, leads to expression or activation of another transcription factor which binds to the promoter operably linked to the second nucleotide sequence and causes expression of the second nucleotide sequence. In alternative aspects, expression of the second nucleotide sequence is repressed until expression of the third nucleotide sequence occurs, and expression of the third nucleotide sequence occurs when the first target binds to the cell surface receptor encoded by the first nucleotide sequence.

Vectors

The cells and compositions of the present disclosure comprise a nucleic acid comprising a first nucleotide sequence and a nucleic acid comprising a second nucleotide sequence. In exemplary instances, the nucleic acid(s) is/are incorporated into a vector. In exemplary aspects, the cells and/or compositions of the present disclosure comprise a vector comprising both the first nucleic acid and the second nucleic acid. In alternative aspects, the cells and/or compositions comprise a first vector and a second vector, wherein the first vector comprises the first nucleic acid and the second vector comprises the second nucleic acid.

In exemplary aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vector is not naturally-occurring as a whole, in exemplary aspects. However, parts of the vectors can be naturally-occurring. The vectors can comprise any type of nucleotides, including, but not limited to DNA and RNA, which can be single-stranded or double-stranded, synthesized or obtained in part from natural sources, and which can contain natural, non-natural or altered nucleotides. The vectors can comprise naturally-occurring or non-naturally-occurring internucleotide linkages, or both types of linkages. In some aspects, the altered nucleotides or non-naturally occurring internucleotide linkages do not hinder the transcription or replication of the vector.

The vector of the present disclosure can be any suitable vector used to transduce, transform, or transfect any host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGTIO, λGTI 1, λZapII (Stratagene), λEMBL4, and λNMI 149, also can be used. Examples of plant expression vectors include pBIOI, pBI101.2, pBI101.3, pBI121 and pBIN19 (Clontech). Examples of animal expression vectors include pEUK-CI, pMAM and pMAMneo (Clontech).

In some aspects, the vector is a viral vector, e.g., a retroviral vector. In exemplary aspects, the viral vector is a vaccinia virus vector, a poxvirus vector, an adenovirus vector, or a herpes simplex virus vector. In exemplary aspects, the viral vector is an adenoassociated virus (AAV) vector. In exemplary aspects, the AAV vector may be of AAV1 serotype, AAV2 serotype, AAV3 serotype, AAV4 serotype, AAV5 serotype, AAV6 serotype, AAV7 serotype, AAV8 serotype, AAV9 serotype, or AAV10 serotype. In exemplary aspects, the viral vector is a lentiviral vector. In exemplary aspects, the viral vector is a lentiviral vector and the first nucleotide sequence and/or second nucleotide sequence are flanked by long terminal repeat (LTR) sequences. In exemplary aspects, the lentiviral vector comprising the first nucleotide sequence and/or second nucleotide sequence are replication-incompetent, comprises a self-inactivating (SIN) deletion in the 3′ LTR, and/or is based on the human immunodeficiency virus-1 (HIV-1), Simian Immunodeficiency virus (SIV), Equine Infectious anemia virus (EIAV), or feline immunodeficiency virus (FIV).

The vectors of the present disclosure can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEI, 2μ plasmid, λ, SV40, bovine papilloma virus, and the like.

In exemplary aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

Cell Surface Receptor

The first nucleotide sequence encodes a cell surface receptor comprising an extracellular domain (ECD) which binds to a first target, a transmembrane domain (TMD), and an intracellular domain (ICD) comprising at least a portion of a T-cell signaling molecule. In exemplary aspects, the ECD of the cell surface receptor comprises a target recognition domain which effects or mediates binding between a target, e.g., the first target, and the cell surface receptor.

In exemplary aspects, the target recognition domain comprises an antigen-binding domain of an antibody or a T-cell receptor (TCR) or B-cell receptor (BCR). In alternative exemplary aspects, the antigen-binding domain comprises an antigen-binding protein, or an antigen-binding portion thereof. In exemplary instances, the antigen-binding domain comprises any of the antigen-binding proteins described herein, including an antibody, antigen-binding fragment thereof, or an antibody protein product. In exemplary aspects, the antigen-binding domain comprises the complementarity determining regions (CDRs) of the light chain variable region of an antibody and the CDRs of the heavy chain variable region of an antibody. In exemplary aspects, the antigen-binding domain comprises CDR1, CDR2, and CDR3 of the light chain variable region of an antibody and CDR1, CDR2, and CDR3 of the heavy chain variable region of an antibody. In exemplary aspects, the antigen-binding domain comprises an antibody, an antigen-binding antibody fragment, or an antibody protein product. Suitable antibodies and antigen binding fragments are known in the art, some of which are described below. In exemplary aspects, the target recognition domain of the ECD comprises a single-chain variable fragment (scFv) or a tandem scFv.

In exemplary aspects, the antigen-binding domain of the ECD comprises the CDRs of the variable domain of the TCR alpha chain and the CDRs of the variable domain of the TCR beta chain. In exemplary aspects, the antigen-binding domain comprises CDR1, CDR2, and CDR3 of the variable domain of the TCR alpha chain and CDR1, CDR2, and CDR3 of the variable domain of the TCR beta chain.

In exemplary aspects, the antigen-binding domain of the ECD comprises the CDRs of the variable domain of a BCR. In exemplary aspects, the BCR comprises a membrane-bound immunoglobulin (Ig) of IgD, IgM, IgA, IgG, or IgE isotype. In exemplary aspects, the antigen-binding domain comprises CDR1, CDR2, and CDR3 of the light chain variable region of the Ig portion of the BCR and CDR1, CDR2, and CDR3 of the heavy chain variable region of the Ig portion of the BCR.

In exemplary aspects, the target recognition domain of the ECD of the cell surface receptor comprises a ligand-binding domain of a cell surface receptor. In exemplary aspects, the ligand-binding domain is of a cell surface receptor which is not naturally expressed by the cell of the present disclosure. For example, the cell of the present disclosure may be an immune cell (e.g., a T-lymphocyte) and the ligand-binding domain is of a cell surface receptor which is not expressed by an immune cell. Thus the ligand-binding domain and the cell surface receptor from which it is derived is considered as exogenous to the immune cell. In exemplary aspects, the ligand-binding domain of the ECD of the cell surface receptor encoded by the first nucleotide sequence is the ligand-binding domain of a G protein-coupled receptor (GPCR), ion channel-linked receptor, or enzyme-linked receptor. The enzyme-linked receptor may be a receptor tyrosine kinase (RTK), a tyrosine kinase associated receptor, a receptor-like tyrosine phosphatase, a receptor serine/threonine kinase, a receptor guanylyl cyclase or a histidine kinase associated receptor. In exemplary instances, the RTK is a growth factor receptor (e.g., epidermal growth factor receptor (EGFR), a platelet-derived growth factor receptor (PDGFR), an insulin receptor, a fibroblast growth factor receptor (FGFR), vascular endothelial growth factor receptor (VEGFR), nerve growth factor receptor (NGFR), hepatocyte growth factor receptor (HGFR), and the like. In exemplary aspects, the ligand binding domain of the ECD of the cell surface receptor encoded by the first nucleotide sequence is the ligand-binding domain of a cytokine receptor. In exemplary aspects, the ligand binding domain of the ECD of the cell surface receptor encoded by the first nucleotide sequence is the ligand-binding domain of SIRP1a. The present disclosure is not limited to these examples.

In exemplary aspects, the target recognition domain of the ECD of the cell surface receptor comprises a receptor-binding domain of a ligand. In exemplary aspects, the receptor-binding domain of the ECD of the cell surface receptor encoded by the first nucleotide sequence is the receptor-binding domain of the ligand that binds to any of the cell surface receptors described herein. In exemplary aspects, the receptor-binding domain of the ECD of the cell surface receptor encoded by the first nucleotide sequence is the receptor-binding domain of epidermal growth factor (EGF), platelet-derived growth factor (PDGF), insulin, a fibroblast growth factor (FGF), vascular endothelial growth factor (VEGF), nerve growth factor (NGF), hepatocyte growth factor (HGF). In exemplary aspects, the receptor-binding domain of the ECD of the cell surface receptor encoded by the first nucleotide sequence is the receptor-binding domain of a hormone, growth factor, cytokine, neurotransmitter, and the like. In exemplary aspects, the receptor-binding domain of the ECD of the cell surface receptor encoded by the first nucleotide sequence is the receptor-binding domain of adenosine, bombesin, bradykinin, endothelin, γ-aminobutyric acid (GABA), hepatocyte growth factor (HGF), melanocortins, neuropeptide Y, opioid peptides, opsins, somatostatin, GH, tachykinins, members of the vasoactive intestinal peptide family, and vasopressin; biogenic amines (e.g., dopamine, epinephrine, norepinephrine, histamine, glutamate (metabotropic effect), glucagon, acetylcholine (muscarinic effect), and serotonin); chemokines; lipid mediators of inflammation (e.g., prostaglandins, prostanoids, platelet-activating factor, and leukotrienes); and peptide hormones (e.g., calcitonin, C5a anaphylatoxin, follicle-stimulating hormone (FSH), gonadotropin-releasing hormone (GnRH), neurokinin, thyrotropin-releasing hormone (TRH), cannabinoids, and oxytocin). In exemplary aspects, the receptor-binding domain of the ECD of the cell surface receptor encoded by the first nucleotide sequence is the receptor-binding domain of CD47. In exemplary aspects, the receptor-binding domain of CD47 comprises SEQ ID NO: 13, or a portion thereof. The present disclosure is not limited to these examples. In exemplary aspects, the receptor-binding domain of the ECD of the cell surface receptor encoded by the first nucleotide sequence is the receptor-binding domain of a cytokine, lymphokine, growth factor, or other hematopoietic factor, including, but not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neurotrophic factor, ciliary neurotrophic factor receptor α, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epithelial-derived neutrophil attractant, glial cell line-derived neurotrophic factor receptor α1, glial cell line-derived neurotrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

In exemplary aspects, the ECD of the cell surface receptor comprises an antibody protein product which binds to the first target and the first target is AFP. In exemplary instances, the antibody protein product is a single-chain Fragment variable (scFv) or a tandem scFv that binds to AFP. In exemplary instances, the scFv comprises one, two, or three heavy chain CDR sequences of an anti-AFP scFV, e.g., one, two, or three of SEQ ID NOs: 79-81. In exemplary aspects, the BsAb comprises a heavy chain variable region sequence of SEQ ID NO: 28. In exemplary instances, the scFv comprises one, two, or three light chain CDR sequences of an anti-AFP scFV, e.g., one, two, or three of SEQ ID NOs: 82-84. In exemplary instances, the scFv comprises a light chain variable region sequence of SEQ ID NO: 29. In exemplary aspects, the anti-AFP scFv comprises both SEQ ID NOs: 28 and 29, optionally, joined together with a linker. In exemplary aspects, the anti-AFP scFv comprises the amino acid sequence of SEQ ID NO: 31.

In exemplary embodiments of the present disclosure, the cell surface receptor encoded by the first nucleotide sequence comprises an ICD comprising at least a portion of a T-cell signaling molecule. As used herein, the term “T-cell signaling molecule” refers to a molecule that is part of the TCR complex, a TCR co-receptor (e.g., CD4, CD8), or a T-cell co-stimulatory molecule. As used herein, “TCR complex” refers to an octomeric complex of variable TCR receptor α and β chains with three dimeric signaling modules CD3δ/ε, CD3γ/ε and CD247ζ/ζ or ζ/η. In exemplary aspects, the T-cell co-stimulatory molecule is selected from the group consisting of: CD3, CD28, Inducible Co-Stimulator (ICOS), CTLA4 (Cytotoxic T-Lymphocyte-Associated protein 4), 4-1BB (also known as CD137), OX40 (also known as CD134), CD27, CD30, DR3, Glucocorticoid-Induced TNFR family Related (GITR) and Herpes Virus Entry Mediator (HVEM). In exemplary instances, the ICD of the cell surface receptor comprises at least a portion of CD3, CD28, 4-1BB, or OX40. In exemplary instances, the ICD of the cell surface receptor comprises at least a portion of CD28, e.g., a CD28 co-stimulatory fragment, such as that comprising the sequence of SEQ ID NO: 56. In exemplary instances, the ICD of the cell surface receptor comprises at least a portion of 4-1BB, e.g., a 4-1BB co-stimulatory fragment, such as that comprising the sequence of SEQ ID NO: 57. In exemplary instances, the ICD of the cell surface receptor comprises at least a portion of OX40, e.g., an OX40 co-stimulatory fragment, such as that comprising the sequence of SEQ ID NO: 58. In exemplary aspects, the ICD comprises a cytoplasmic portion of CD3-ζ. In exemplary aspects, the ICD of the cell surface receptor comprises an amino acid sequence of SEQ ID NO: 36. In some aspects, the ICD of the cell surface receptor encoded by the first nucleotide sequence comprises at least a portion of two different T-cell signaling molecules. The ICD comprises the cytoplasmic portion of each of the two T-cell signaling molecules in some aspects. In exemplary instances each of the two T-cell signaling molecules of the ICD is independently selected from the group consisting of: CD3, CD28, 4-1BB, and OX40. In some aspects, the ICD comprises the cytoplasmic portion of CD3-ζ and the cytoplasmic portion of CD28. In exemplary aspects, the ICD of the cell surface receptor comprises an amino acid sequence of SEQ ID NOs: 35 and 36.

The TMD of the cell surface receptor encoded by the first nucleotide sequence may comprise a TMD of any cell surface protein known in the art, except for the TMD of any of TCRs (such as the TCR α chain, TCR β chain, TCR δ chain, or TCR γ chain). The TMD of the cell surface receptor encoded by the first nucleotide sequence may comprise the TMD of any of the cell surface receptors, T-cell signaling molecules, or T-cell co-stimulatory molecules described herein. The TMD of the cell surface receptor encoded by the first nucleotide sequence may comprise the TMD of an immunoglobulin or a BCR. In exemplary aspects, the TMD of the cell surface receptor comprises at least a portion of a T-cell signaling molecule. In exemplary aspects, the TMD of the cell surface receptor comprises at least a portion of a T-cell signaling molecule which is different from the T-cell signaling molecule of the ICD. In alternative aspects, the TMD of the cell surface receptor comprises at least a portion of a T-cell signaling molecule which is the same as the T-cell signaling molecule of the ICD. In exemplary instances, the TMD comprises at least a portion of CD28, 4-1BB, or OX40. In exemplary instances, the TMD comprises at least a portion of a CD28 TMD, e.g., comprising the amino acid sequence of SEQ ID NO: 34.

The cell surface receptor encoded by the first nucleotide sequence may further comprise additional portions of a T-cell signaling molecule. In exemplary aspects, the cell surface receptor further comprises a portion of the ECD of CD28. In exemplary aspects, the portion of the ECD of CD28 comprises the amino acid sequence of SEQ ID NO: 33. In exemplary aspects, the cell surface receptor comprises an scFv, at least a portion of the ECD of CD28 (e.g., SEQ ID NO: 33), the TMD of CD28 (e.g., SEQ ID NO: 34), and the cytoplasmic portion of CD3-ζ (e.g., SEQ ID NO: 36) and the cytoplasmic portion of CD28 (e.g., SEQ ID NO: 35). In exemplary aspects, the cell surface receptor comprises the amino acid sequences of SEQ ID NOs: 33-36 as shown in Tables 3 and 5. In exemplary aspects, the ECD comprises an anti-AFP scFv. In exemplary aspects, the BsAb comprises a heavy chain variable region sequence of SEQ ID NO: 28 or a light chain variable region sequence of SEQ ID NO: 29 or both SEQ ID NOs: 28 and 29. In exemplary aspects, the anti-AFP scFv comprises the amino acid sequence of SEQ ID NO: 31.

Antigen-Binding Proteins

In exemplary embodiments, the second nucleotide sequence encodes an antigen-binding protein. The term “antigen-binding protein” refers to any molecule which is capable of binding to a target protein, e.g., an antigen. The antigen-binding proteins can take any one of many forms of antigen-binding proteins known in the art. In exemplary embodiments, the antigen-binding proteins of the present disclosure take the form of an antibody, or antigen-binding antibody fragment, or an antibody protein product.

The term “antibody” refers to a protein comprising two heavy chains and two light chains, each of which comprises a variable region and a constant region. The variable regions are responsible for binding antigen. Each variable region contains three highly variable loops (hypervariable loops), called the complementarity determining regions (CDRs). Accordingly, an antibody comprises two light chains, each of which comprises light chain (LC) CDRs (LC-CDR1, LC-CDR2, and LC-CDR3), and two heavy chains, each of which comprises heavy chain (HC) CDRs, (HC-CDR1, HC-CDR2, and HC-CDR3). The three CDRs of the heavy and light chains are interposed between flanking stretches known as framework regions (FRs), which are more conserved than the CDRs and form a scaffold to support the hypervariable loops.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR DEFINITIONS

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The constant regions of the heavy and light chains are not involved in antigen-binding, but exhibit various effector functions. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). With regard to the present disclosure, the antibody encoded by the second nucleotide may be any type of immunoglobulin known in the art. In exemplary aspects, the antibody is an antibody of isotype IgA, IgD, IgE, IgG, or IgM.

The antibody may be a monoclonal antibody or a polyclonal antibody. In exemplary embodiments, the antibody is a naturally-occurring antibody, e.g., an antibody isolated and/or purified from a mammal, e.g., mouse, rabbit, goat, horse, chicken, hamster, human, and the like. In this regard, the antibody may be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody.

In exemplary embodiments, the antibody is not a naturally-occurring antibody. In exemplary instances, the antibody is a genetically-engineered antibody, e.g., a humanized antibody, a chimeric antibody, an antibody which includes portions of CDR sequences, a humaneered antibody, and the like. Genetic engineering techniques also provide the ability to make fully human antibodies in a non-human source.

In some aspects, the antibody is a chimeric antibody. The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)).

In some aspects, the antibody is a humanized antibody. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence. “Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Use of the terms “chimeric or humanized” herein is not meant to be mutually exclusive, and rather, is meant to encompass chimeric antibodies, humanized antibodies, and chimeric antibodies that have been further humanized. Except where context otherwise indicates, statements about (properties of, uses of, testing of, and so on) chimeric antibodies of the invention apply to humanized antibodies of the invention, and statements about humanized antibodies of the invention pertain also to chimeric antibodies. Likewise, except where context dictates, such statements also should be understood to be applicable to antibodies and antigen binding fragments of such antibodies of the invention.

In some aspects, the antibody is a human antibody. The human antibody may be a naturally occurring antibody or a genetically engineered antibody comprising CDR and/or framework sequences generated from human germ-line immunoglobulin genes through recombination and/or splicing, but had not been generated naturally yet). One advantage of using a human antibody is to reduce the immune response against the immune cells transduced with nucleic acids encoding the antibody in human patients receiving or producing the antibodies.

Human antibodies can be generated in several ways. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

In some embodiments, the antibody moiety is fully human, semi-synthetic with human antibody framework regions. In some embodiments, an antigen-binding fragment may comprise one or more CDRs from a particular human antibody grafted to a framework region from one or more different human antibodies.

In some aspects, the antibody is a humaneered™ antibody. Humaneering technology is a proprietary method of KaloBios Pharmaceuticals, Inc. (San Francisco, Calif.) for converting non-human antibodies into engineered human antibodies. Humaneered™ antibodies are high affinity, and highly similar to human germline antibody sequences.

Antigen Binding Fragments

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects of the present disclosure, the antigen-binding protein of the present disclosure is an antigen-binding fragment of an antibody (a.k.a., antigen-binding antibody fragment, antigen-binding fragment, antigen-binding portion). In exemplary instances, the antigen-binding antibody fragment is a Fab fragment or a F(ab′)₂ fragment. In exemplary aspects, the second nucleotide sequence encodes an antigen-binding fragment of an antibody.

Antibody Protein Products

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the antigen-binding protein of the present disclosure comprises, consists essentially of, or consists of any one of these antibody protein products. In exemplary aspects, the antigen-binding protein of the present disclosure comprises, consists essentially of, or consists of any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate.

In exemplary instances, the antigen-binding protein of the present disclosure is an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In some embodiments, the antibody is in monomeric form, while in other embodiments, the antibody is conjugated to one or more antibodies (e.g., each of which recognize the same epitope of the first antibody). Accordingly, in some aspects, the antibody is in polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In some aspects, the antibody is a bispecific or trispecific antibody.

Bispecific antibodies (BsAb) are molecules comprising two single-chain Fv fragments joined via a glycine-serine linker using recombinant methods. The V light-chain (V_{L}) and V heavy-chain (V_{H}) domains of two antibodies of interest in exemplary embodiments are isolated using standard PCR methods. The V_{L} and V_{H} cDNA's obtained from each hybridoma are then joined to form a single-chain fragment in a two-step fusion PCR. Bispecific fusion proteins are prepared in a similar manner. Bispecific single-chain antibodies and bispecific fusion proteins are antibody substances included within the scope of the present invention. Exemplary bispecific antibodies are taught in U.S. Patent Application Publication No. 2005-0282233A1 and International Patent Application Publication No. WO 2005/087812, both applications of which are incorporated herein by reference in their entirety. In exemplary aspects, the bispecific or trispecific antibody binds to an epitope as further described herein under the section entitled “Epitopes.” Methods of making bispecific or trispecific antibodies are known in the art. See, for example, Marvin and Zhu, Acta Pharmacologica Sinica 26: 649-658 (2005) and U.S. Pat. No. 6,551,592.

In embodiments of the present disclosure, the second nucleotide sequence encodes an antigen-binding protein (e.g., an antibody protein product) which binds to a second target and a third target as further described herein. In exemplary aspects, the second target is different from the third target. Accordingly, the antigen-binding protein is bispecific antigen-binding protein or multispecific antigen-binding protein.

Targets

With regard to the present disclosure, the cell surface receptor encoded by the first nucleotide sequence binds to a first target and the antigen-binding protein encoded by the second nucleotide sequence binds to a second target and third target. As used herein, the term “target” refers to any biomolecule that is bound by the cell surface receptor or antigen-binding protein. The target may be considered as a “ligand” or an “antigen”. Accordingly, the target may be any ligand of a cell surface receptor described herein.

In exemplary embodiments, the first target and/or third target is/are a ligand or antigen that is representative of a disease. In exemplary aspects, the target is expressed by a diseased cell, and, in exemplary instances, the target is characteristic of the diseased cell. In exemplary aspects, the target is predominantly, specifically, or exclusively expressed by a diseased cell, wherein the expression of the target by the diseased cell is relative to expression by normal, undiseased, or healthy cells. In exemplary aspects, the diseased cell expresses the target at a level which is at least 10% or more than the level of expression of the target by a normal, undiseased, or healthy cell. In exemplary aspects, the diseased cell expresses the target at a level which is at least 20% or more than the level of expression of the target by a normal, undiseased, or healthy cell. In exemplary aspects, the diseased cell expresses the target at a level which is at least 30% or more than the level of expression of the target by a normal, undiseased, or healthy cell. In exemplary aspects, the diseased cell expresses the target at a level which is at least 40% or more than the level of expression of the target by a normal, undiseased, or healthy cell. In exemplary aspects, the diseased cell expresses the target at a level which is at least 50% or more than the level of expression of the target by a normal, undiseased, or healthy cell. In exemplary aspects, the diseased cell expresses the target at a level which is at least 60% or more than the level of expression of the target by a normal, undiseased, or healthy cell. In exemplary aspects, the diseased cell expresses the target at a level which is at least 70% or more than the level of expression of the target by a normal, undiseased, or healthy cell. In exemplary aspects, the diseased cell expresses the target at a level which is at least 80% or more than the level of expression of the target by a normal, undiseased, or healthy cell.

In exemplary aspects, the disease is a tumor or cancer. The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In exemplary aspects, the tumor is a solid tumor.

In exemplary aspects, one or more of the first, second, and third targets is/are an antigen expressed by a tumor or cancer cell. In exemplary aspects, the antigen expressed by a tumor or cancer cell is an antigen derived from a viral protein, an antigen derived from point mutations, or an antigen encoded by a cancer-germline gene. In exemplary aspects, the antigen expressed by a tumor or cancer cell is p53, KRAS, NRAS, MAGEA, MAGEB, MAGEC, BAGE, GAGE, LAGE/NY-ESO1, SSX, tyrosinase, gp100/pmeI17, Melan-A/MART-1, gp75/TRP1, TRP2, CEA, RAGE-1, HER2/NEU, WT1, ROR1, ROR2, BCMA, GPRC5D, FcRL5, AFP, HPV16-E7, PRAME, EBV-LMP2A, PSA, FoxP3, GPC3, Histone H3.3, or CD19. See, e.g., International Patent Application Publication Nos. WO2017/124001; WO2017/096120; WO2017/070608; WO2017/066136; WO2017/015634; WO2016/210365; WO2016/210129; WO2016/201124; WO2016/191246; WO2016/187220; WO2016/187216; WO2016/161390; WO2016/154047; WO2016142768; WO2016/090312; WO2016/090320; WO2016/090327; WO2016/090329; WO2016/090337; WO2015/070078; WO2015/070061; and WO2010/036443, each of which are incorporated herein by reference.

In exemplary aspects, the disease is an infectious disease and the target is an antigen of a pathogen. The pathogen in exemplary aspects, is a bacterial pathogen or a viral pathogen. In exemplary aspects, the infectious disease or the source thereof is any of those listed in Table 2.

TABLE 2

| Disease | Pathogen/Source of Disease |
|---|---|
| Acinetobacter infections | Acinetobacter baumannii |
| Actinomycosis | Actinomyces israelii, |
| | Actinomyces gerencseriae and |
| | Propionibacterium propionicus |
| African sleeping sickness | Trypanosoma brucei |
| (African trypanosomiasis) | |
| Acquired immunodeficiency | HIV (Human immunodeficiency |
| syndrome (AIDS) | virus) |
| Amebiasis | Entamoeba histolytica |
| Anaplasmosis | Anaplasma species |
| Angiostrongyliasis | Angiostrongylus |
| Anisakiasis | Anisakis |
| Anthrax | Bacillus anthracis |
| Arcanobacterium | Arcanobacterium haemolyticum |
| haemolyticum | |
| infection | |
| Argentine hemorrhagic fever | Junin virus |
| Ascariasis | Ascaris lumbricoides |
| Aspergillosis | Aspergillus species |
| Astrovirus infection | Astroviridae family |
| Babesiosis | Babesia species |
| Bacillus cereus infection | Bacillus cereus |
| Bacterial pneumonia | multiple bacteria |
| Bacterial vaginosis | List of bacterial vaginosis |
| | microbiota |
| Bacteroides infection | Bacteroides species |
| Balantidiasis | Balantidium coli |
| Bartonellosis | Bartonella |
| Baylisascaris infection | Baylisascaris species |
| BK virus infection | BK virus |
| Black piedra | Piedraia hortae |
| Blastocystosis | Blastocystis species |
| Blastomycosis | Blastomyces dermatitidis |
| Bolivian hemorrhagic fever | Machupo virus |
| Botulism (and Infant botulism) | Clostridium botulinum; |
| | Note: |
| | Botulism is not an infection by |
| | Clostridium botulinum but caused |
| | by the intake |
| | of botulinum toxin. |
| Brazilian hemorrhagic fever | Sabiá virus |
| Brucellosis | Brucella species |
| Bubonic plague | the bacterial family |
| | Enterobacteriaceae |
| Burkholderia infection | usually Burkholderia cepacia and |
| | other Burkholderia species |
| Buruli ulcer | Mycobacterium ulcerans |
| Calicivirus infection | Caliciviridae family |
| (Norovirus and Sapovirus) | |
| Campylobacteriosis | Campylobacter species |
| Candidiasis (Moniliasis; Thrush) | usually Candida albicans and other |
| | Candida species |
| Capillariasis | Intestinal disease by |
| | Capillaria philippinensis, hepatic |
| | disease by Capillaria hepatica and |
| | pulmonary disease by |
| | Capillaria aerophila |
| Carrion's disease | Bartonella bacilliformis |
| Cat-scratch disease | Bartonella henselae |
| Cellulitis | usually Group A Streptococcus and |
| | Staphylococcus |
| Chagas Disease (American | Trypanosoma cruzi |
| trypanosomiasis) | |
| Chancroid | Haemophilus ducreyi |
| Chickenpox | Varicella zoster virus (VZV) |
| Chikungunya | Alphavirus |
| Chlamydia | Chlamydia trachomatis |
| Chlamydophila pneumoniae | Chlamydophila pneumoniae |
| infection | |
| (Taiwan acute | |
| respiratory agent or | |
| TWAR) | |
| Cholera | Vibrio cholerae |
| Chromoblastomycosis | usually Fonsecaea pedrosoi |
| Chytridiomycosis | Batrachochytrium dendrabatidis |
| Clonorchiasis | Clonorchis sinensis |
| Clostridium difficile colitis | Clostridium difficile |
| Coccidioidomycosis | Coccidioides immitis and |
| | Coccidioides posadasii |
| Colorado tick fever (CTF) | Colorado tick fever virus (CTFV) |
| Common cold (Acute viral | usually rhinoviruses and |
| rhinopharyngitis; Acute coryza) | coronaviruses |
| Creutzfeldt-Jakob disease (CJD) | PRNP |
| Crimean-Congo hemorrhagic | Crimean-Congo hemorrhagic |
| fever (CCHF) | fever virus |
| Cryptococcosis | Cryptococcus neoformans |
| Cryptosporidiosis | Cryptosporidium species |
| Cutaneous larva migrans (CLM) | usually Ancylostoma braziliense, |
| | multiple other parasites |
| Cyclosporiasis | Cyclospora cayetanensis |
| Cysticercosis | Taenia solium |
| Cytomegalovirus infection | Cytomegalovirus |
| Dengue fever | Dengue viruses (DEN-1, DEN-2, |
| | DEN-3 and DEN-4) - |
| | Flaviviruses |
| Desmodesmus infection | Green algae Desmodesmus armatus |
| Dientamoebiasis | Dientamoeba fragilis |
| Diphtheria | Corynebacterium diphtheriae |
| Diphyllobothriasis | Diphyllobothrium |
| Dracunculiasis | Dracunculus medinensis |
| Ebola hemorrhagic fever | Ebolavirus (EBOV) |
| Echinococcosis | Echinococcus species |
| Ehrlichiosis | Ehrlichia species |
| Enterobiasis (Pinworm infection) | Enterobius vermicularis |
| Enterococcus infection | Enterococcus species |
| Enterovirus infection | Enterovirus species |
| Epidemic typhus | Rickettsia prowazekii |
| Erythema infectiosum | Parvovirus B19 |
| (Fifth disease) | |
| Exanthem subitum (Sixth disease) | Human herpesvirus 6 (HHV-6) and |
| | Human herpesvirus 7 (HHV-7) |
| Fasciolasis | Fasciola hepatica and |
| | Fasciola gigantica |
| Fasciolopsiasis | Fasciolopsis buski |
| Fatal familial insomnia (FFI) | PRNP |
| Filariasis | Filarioidea superfamily |
| Food poisoning by | Clostridium perfringens |
| Clostridium perfringens | |
| Free-living amebic infection | multiple |
| Fusobacterium infection | Fusobacterium species |
| Gas gangrene | usually Clostridium perfringens; |
| (Clostridial myonecrosis) | other Clostridium species |
| Geotrichosis | Geotrichum candidum |
| Gerstmann-Sträussler-Scheinker | PRNP |
| syndrome (GSS) | |
| Giardiasis | Giardia lamblia |
| Glanders | Burkholderia mallei |
| Gnathostomiasis | Gnathostoma spinigerum and |
| | Gnathostoma hispidum |
| Gonorrhea | Neisseria gonorrhoeae |
| Granuloma inguinale | Klebsiella granulomatis |
| (Donovanosis) | |
| Group A streptococcal infection | Streptococcus pyogenes |
| Group B streptococcal infection | Streptococcus agalactiae |
| Haemophilus influenzae infection | Haemophilus influenzae |
| Hand, foot and mouth disease | Enteroviruses, mainly Coxsackie A |
| (HFMD) | virus and Enterovirus 71 (EV71) |
| Hantavirus Pulmonary Syndrome | Sin Nombre virus |
| (HPS) | |
| Heartland virus disease | Heartland virus |
| Helicobacter pylori infection | Helicobacter pylori |
| Hemolytic-uremic syndrome | Escherichia coli O157:H7, O111 |
| (HUS) | and O104:H4 |
| Hemorrhagic fever with renal | Bunyaviridae family |
| syndrome (HFRS) | |
| Hepatitis A | Hepatitis A virus |
| Hepatitis B | Hepatitis B virus |
| Hepatitis C | Hepatitis C virus |
| Hepatitis D | Hepatitis D Virus |
| Hepatitis E | Hepatitis E virus |
| Herpes simplex | Herpes simplex virus 1 and 2 |
| | (HSV-1 and HSV-2) |
| Histoplasmosis | Histoplasma capsulatum |
| Hookworm infection | Ancylostoma duodenale and |
| | Necator americanus |
| Human bocavirus infection | Human bocavirus (HBoV) |
| Human ewingii ehrlichiosis | Ehrlichia ewingii |
| Human granulocytic | Anaplasma phagocytophilum |
| anaplasmosis (HGA) | |
| Human metapneumovirus | Human metapneumovirus |
| infection | (hMPV) |
| Human monocytic ehrlichiosis | Ehrlichia chaffeensis |
| Human papillomavirus | Human papillomavirus (HPV) |
| (HPV) infection | |
| Human parainfluenza virus | Human parainfluenza viruses |
| infection | (HPIV) |
| Hymenolepiasis | Hymenolepis nana and |
| | Hymenolepis diminuta |
| Epstein-Barr virus infectious | Epstein-Barr virus (EBV) |
| mononucleosis (Mono) | |
| Influenza (flu) | Orthomyxoviridae family |
| Isosporiasis | Isospora belli |
| Kawasaki disease | unknown; evidence supports |
| | that it is infectious |
| Keratitis | multiple |
| Kingella kingae infection | Kingella kingae |
| Kuru | PRNP |
| Lassa fever | Lassa virus |
| Legionellosis (Legionnaires' | Legionella pneumophila |
| disease) | |
| Legionellosis (Pontiac fever) | Legionella pneumophila |
| Leishmaniasis | Leishmania species |
| Leprosy | Mycobacterium leprae and |
| | Mycobacterium lepromatosis |
| Leptospirosis | Leptospira species |
| Listeriosis | Listeria monocytogenes |
| Lyme disease | Borrelia burgdorferi, |
| (Lyme borreliosis) | Borrelia garinii, |
| | and Borrelia afzelii |
| Lymphatic filariasis | Wuchereria bancrofti and |
| (Elephantiasis) | Brugia malayi |
| Lymphocytic choriomeningitis | Lymphocytic choriomeningitis |
| | virus (LCMV) |
| Malaria | Plasmodium species |
| Marburg hemorrhagic fever | Marburg virus |
| (MHF) | |
| Measles | Measles virus |
| Middle East respiratory | Middle East respiratory syndrome |
| syndrome (MERS) | coronavirus |
| Melioidosis | Burkholderia pseudomallei |
| (Whitmore's disease) | |
| Meningitis | multiple |
| Meningococcal disease | Neisseria meningitidis |
| Metagonimiasis | usually Metagonimus yokagawai |
| Microsporidiosis | Microsporidia phylum |
| Molluscum contagiosum (MC) | Molluscum contagiosum virus |
| | (MCV) |
| Monkeypox | Monkeypox virus |
| Mumps | Mumps virus |
| Murine typhus (Endemic typhus) | Rickettsia typhi |
| Mycoplasma pneumonia | Mycoplasma pneumoniae |
| Mycetoma (disambiguation) | numerous species of bacteria |
| | (Actinomycetoma) |
| | and fungi (Eumycetoma) |
| Myiasis | parasitic dipterous fly larvae |
| Neonatal conjunctivitis | most commonly |
| (Ophthalmia neonatorum) | Chlamydia trachomatis and |
| | Neisseria gonorrhoeae |
| Norovirus (children and babies) | Norovirus |
| (New) Variant Creutzfeldt-Jakob | PRNP |
| disease (vCJD, nvCJD) | |
| Nocardiosis | usually Nocardia asteroides and |
| | other Nocardia species |
| Onchocerciasis (River blindness) | Onchocerca volvulus |
| Opisthorchiasis | Opisthorchis viverrini and |
| | Opisthorchis felineus |
| Paracoccidioidomycosis (South | Paracoccidioides brasiliensis |
| American blastomycosis) | |
| Paragonimiasis | usually Paragonimus westermani |
| | and other Paragonimus species |
| Pasteurellosis | Pasteurella species |
| Pediculosis capitis (Head lice) | Pediculus humanus capitis |
| Pediculosis corporis (Body lice) | Pediculus humanus corporis |
| Pediculosis pubis (Pubic lice, | Phthirus pubis |
| Crab lice) | |
| Pelvic inflammatory disease | multiple |
| (PID) | |
| Pertussis (Whooping cough) | Bordetella pertussis |
| Plague | Yersinia pestis |
| Pneumococcal infection | Streptococcus pneumoniae |
| Pneumocystis pneumonia (PCP) | Pneumocystis jirovecii |
| Pneumonia | multiple |
| Poliomyelitis | Poliovirus |
| Prevotella infection | Prevotella species |
| Primary amoebic | usually Naegleria fowleri |
| meningoencephalitis (PAM) | |
| Progressive multifocal | JC virus |
| leukoencephalopathy | |
| Psittacosis | Chlamydophila psittaci |
| Q fever | Coxiella burnetii |
| Rabies | Rabies virus |
| Relapsing fever | Borrelia hermsii, |
| | Borrelia recurrentis, and |
| | other Borrelia species |
| Respiratory syncytial virus | Respiratory syncytial virus (RSV) |
| infection | |
| Rhinosporidiosis | Rhinosporidium seeberi |
| Rhinovirus infection | Rhinovirus |
| Rickettsial infection | Rickettsia species |
| Rickettsialpox | Rickettsia akari |
| Rift Valley fever (RVF) | Rift Valley fever virus |
| Rocky Mountain spotted fever | Rickettsia rickettsii |
| (RMSF) | |
| Rotavirus infection | Rotavirus |
| Rubella | Rubella virus |
| Salmonellosis | Salmonella species |
| SARS (Severe Acute | SARS coronavirus |
| Respiratory Syndrome) | |
| Scabies | Sarcoptes scabiei |
| Schistosomiasis | Schistosoma species |
| Sepsis | multiple |
| Shigellosis (Bacillary | Shigella species |
| dysentery) | |
| Shingles (Herpes zoster) | Varicella zoster virus (VZV) |
| Smallpox (Variola) | Variola major or Variola minor |
| Sporotrichosis | Sporothrix schenckii |
| Staphylococcal food poisoning | Staphylococcus species |
| Staphylococcal infection | Staphylococcus species |
| Strongyloidiasis | Strongyloides stercoralis |
| Subacute sclerosing | Measles virus |
| panencephalitis | |
| Syphilis | Treponema pallidum |
| Taeniasis | Taenia species |
| Tetanus (Lockjaw) | Clostridium tetani |
| Tinea barbae (Barber's itch) | usually Trichophyton species |
| Tinea capitis (Ringworm of | usually Trichophyton tonsurans |
| the Scalp) | |
| Tinea corporis (Ringworm of | usually Trichophyton species |
| the Body) | |
| Tinea cruris (Jock itch) | usually Epidermophyton floccosum, |
| | Trichophyton rubrum, |
| | and Trichophyton mentagrophytes |
| Tinea manum (Ringworm of | Trichophyton rubrum |
| the Hand) | |
| Tinea nigra | usually Hortaea werneckii |
| Tinea pedis (Athlete's foot) | usually Trichophyton species |
| Tinea unguium | usually Trichophyton species |
| (Onychomycosis) | |
| Tinea versicolor | Malassezia species |
| (Pityriasis versicolor) | |
| Toxocariasis (Ocular Larva | Toxocara canis or Toxocara cati |
| Migrans (OLM)) | |
| Toxocariasis (Visceral Larva | Toxocara canis or Toxocara cati |
| Migrans (VLM)) | |
| Trachoma | Chlamydia trachomatis |
| Toxoplasmosis | Toxoplasma gondii |
| Trichinosis | Trichinella spiralis |
| Trichomoniasis | Trichomonas vaginalis |
| Trichuriasis (Whipworm | Trichuris trichiura |
| infection) | |
| Tuberculosis | usually |
| | Mycobacterium tuberculosis |
| Tularemia | Francisella tularensis |
| Typhoid fever | Salmonella enterica subsp. |
| | enterica, serovar typhi |
| Typhus fever | Rickettsia |
| Ureaplasma urealyticum | Ureaplasma urealyticum |
| infection | |
| Valley fever | Coccidioides immitis or |
| | Coccidioides posadasii.[1] |
| Venezuelan equine encephalitis | Venezuelan equine |
| | encephalitis virus |
| Venezuelan hemorrhagic fever | Guanarito virus |
| Vibrio vulnificus infection | Vibrio vulnificus |
| Vibrio parahaemolyticus enteritis | Vibrio parahaemolyticus |
| Viral pneumonia | multiple viruses |
| West Nile Fever | West Nile virus |
| White piedra (Tinea blanca) | Trichosporon beigelii |
| Yersinia pseudotuberculosis | Yersinia pseudotuberculosis |
| infection | |
| Yersiniosis | Yersinia enterocolitica |
| Yellow fever | Yellow fever virus |
| Zygomycosis | Mucorales order (Mucormycosis) |
| | and Entomophthorales order |
| | (Entomophthoramycosis) |

In exemplary aspects, the first target is an antigen expressed by a tumor or cancer cell or an antigen of a pathogen. In exemplary aspects, the third target is an antigen expressed by a tumor or cancer cell or an antigen of a pathogen. In exemplary instances, each of the first target and the third target is an antigen expressed by a tumor or cancer cell or an antigen of a pathogen and optionally, the first target is different from the third target. In exemplary aspects, the first target is expressed by the same type of cell expressing the third target.

In exemplary aspects, each of the first target and the third target is an antigen expressed by a tumor or cancer cell. Optionally, the tumor or cancer cell is a cell of a solid tumor. In exemplary aspects, the first target and/or third target is present on the surface of the cells of the solid tumor.

In some aspects, the first target and/or third target is a peptide bound to an MHC molecule. The first and/or third target may be a peptide of an antigen expressed by a tumor or cancer cell, wherein the antigen is p53, KRAS, NRAS, MAGEA, MAGEB, MAGEC, BAGE, GAGE, LAGE/NY-ESO1, SSX, tyrosinase, gp100/pmeI17, Melan-A/MART-1, gp75/TRP1, TRP2, CEA, RAGE-1, HER2/NEU, WT1, AFP, HPV16-E7, PRAME, EBV-LMP2A, PSA, FoxP3, or Histone H3.3, wherein the peptide is bound to the MHC molecule. In exemplary aspects, the first target is an alpha fetoprotein (AFP) peptide bound to an MHC molecule. In some aspects, the first and/or third target may be a peptide of an antigen expressed by a pathogen, such as a virus, wherein the antigen is HPV16-E7, EBV-LMP2A, or HIV-RT, wherein the peptide is bound to the MHC molecule. In some aspects, the cell surface receptor (CSR) encoded by the first nucleotide binds to the complex formed by the first target peptide and the MHC molecule with a higher binding affinity than to the binding affinity between the CSR and the peptide without MHC or between the CSR and the MHC without the peptide. In some embodiments, the CSR specifically binds to the complex formed by the first target peptide and the MHC molecule, but does not specifically bind to the peptide without MHC or the MHC without the peptide. In some aspects, the antigen-binding protein encoded by the second nucleotide binds to the complex formed by the third target peptide and the MHC molecule with a higher binding affinity than the binding affinity between the antigen-binding protein and the peptide without MHC or between the antigen-binding protein and the MHC without the peptide. In some embodiments, the antigen-binding protein specifically binds to the complex formed by the third target peptide and the MHC molecule, but does not specifically bind to the peptide without MHC or the MHC without the peptide.

The MHC molecule may be an MHC Class I molecule or an MHC Class II molecule. In exemplary aspects, the MHC Class I molecule is a human MHC Class I molecule, e.g., an HLA-A molecule, an HLA-B molecule, or an HLA-C molecule. In exemplary instances, the MHC Class I molecule is an HLA-A*02 molecule.

In some aspects, the third target is selected from the group consisting of: GPC3, CD47, MUC16, CD19, CD20, CD22, EpCAM, EGFR, HER2 CEA, PSMA, AFP, PSA, BCMA, FCRL5, NY-ESO, HPV16, and FoxP3. In exemplary aspects, the third target is GPC3. In exemplary aspects, the third target is CD47, CD19, or PSMA.

In exemplary aspects, the second target is expressed on the surface of an immune cell. In exemplary aspects, the second target is a Cluster of Differentiation (CD) antigen, a cytokine receptor, a chemokine receptor, a T-cell signaling molecule. In exemplary aspects, the CD antigen is selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31,CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79α, CD79β, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182. In exemplary aspects, the immune cell surface antigen is CD3 or CD16a. In exemplary aspects, the cytokine receptor is an IL-2 receptor, IL-3 receptor, IL-4 receptor, IL-5 receptor, IL-6 receptor, IL-7 receptor, IL-9 receptor, IL-11 receptor, IL-13 receptor, G-CSF receptor, IL-15 receptor, GM-CSF receptor, OSM receptor, IFN-γ receptor, IFN-α receptor, IFN-β receptor, TNF-α receptor, TNF-β receptor, LT-β, CD40L, FasL, CD27L, CD30L, 4-BB1, IL-1 RA, MIF receptor, IL-10 receptor, IL-16 receptor, IL-17 receptor, or IL-18 receptor. In exemplary instances, the chemokine receptor is the IL-8 receptor, Mig receptor, IP-10 receptor, SDF-1α/β receptor, MIP-1α receptor, MIP-1β receptor, MDC receptor, TECK receptor, TARC receptor, RANTES receptor, DC-CK1 receptor, MIP3α receptor, MCP1 receptor, MCP2 receptor, MCP3 receptor, MCP4 receptor, Eotaxin-2 receptor, MIP5 receptor, MPIF 1 receptor, 6Ckine receptor, lymphotactin receptor, or fractalkine receptor.

In exemplary aspects, the second target is expressed on the surface of an T-cell. In exemplary aspects, the second target is selected from the group consisting of: CD2, CD3, CD4, CD5, CD6, CD7, CD8, CD9, CD10, CD11a, CD11, CD15, CD18, CD25, CD26, CD27, CD28, CD29, CD30, CD31, CD37, CD38, CD43, CD44, CD45, CD45RO, CD45RA, CD45RB, CD46, CD47, CD48, CD49a, CD49e, CD49f, CD50, CD52, CD53, CD54, CD55, CD57, CD58, CD59, CDw60, CD62I, CD69, CD70, CD71, CD73, CD75, CD76, CD81, CD82, CD87, CD88, CD89, CD90, CD94, CD96, CD97, CD98, CD99, CD100, CD101, CD104, CD107a, CD107b, CD109, CD120, CD121, CD122, CD124, CD127, CDw128, CD132, CD134, CDw137, CD148, CD150, CD152, CD153, CD154, CD161, CD165, and CD166. In exemplary aspects, the second target is expressed on the surface of only T-cells. In exemplary aspects, the second target binds to or engages T-cells. In exemplary aspects, the second target is a component of the TCR complex, e.g., the TCR. In exemplary aspects, the second target is CD3.

In exemplary aspects, the second target is expressed on the surface of a natural killer (NK) cell. In exemplary aspects, the second target is selected from the group consisting of: CD2, CD11b, CD16, CD27, CD30, CD39, CD45, CD56, CD57, CD58, CD59, CD62L, CD69, CD87, CD94, CD96, CD98, CD122, CD132, CD158a, CD158b, and CD161. In exemplary aspects, the second target is expressed on the surface of only NK cells. In exemplary aspects, the second target binds to or engages NK cells. In exemplary aspects, the second target is CD16, e.g., CD16a.

In exemplary aspects, the second target is CD3 and the third target is GPC3, PSMA, CD47, or CD19. In exemplary aspects, the second target is CD16a and the third target is GPC3, PSMA, CD47, or CD19. In exemplary instances, the second nucleotide sequence encodes an antigen-binding protein comprising, consisting essentially of, or consisting of a bispecific antibody (BsAb) that binds to CD3 and GPC3, PSMA, CD47, or CD19. In exemplary aspects, the BsAb comprises one, two, or three heavy chain CDR sequences of an anti-CD3 scFv, e.g., one, two, or three of SEQ ID NOs: 50-52 and/or one, two, or three light chain CDR sequences of an anti-CD3 scFv, e.g., one, two, or three of SEQ ID NOs: 53-55. In exemplary aspects, the BsAb comprises all six CDR sequences of an anti-CD3 scFv, e.g., all six of SEQ ID NOs: 50-55. In exemplary aspects, the BsAb comprises a heavy chain variable region sequence of SEQ ID NO: 41 or a light chain variable region sequence of SEQ ID NO: 43 or both SEQ ID NOs: 41 and 43. In exemplary aspects, the anti-CD3 scFv comprises the amino acid sequence of SEQ ID NO: 24. In exemplary instances, the second nucleotide sequence encodes an antigen-binding protein comprising, consisting essentially of, or consisting of a bispecific antibody (BsAb) that binds to CD3 and GPC3. In exemplary aspects, the BsAb comprises one, two, or three heavy chain CDR sequences of an anti-GPC3 scFv, e.g., one, two, or three of SEQ ID NOs: 15-17 and/or one, two, or three light chain CDR sequences of an anti-GPC3 scFv, e.g., one, two, or three of SEQ ID NOs: 18-20. In exemplary aspects, the BsAb comprises all six CDR sequences of an anti-GPC3 scFv, e.g., all six of SEQ ID NOs: 15-20. In exemplary aspects, the BsAb comprises a heavy chain variable region sequence of SEQ ID NO: 21 or a light chain variable region sequence of SEQ ID NO: 22 or both SEQ ID NOs: 21 and 22. In exemplary aspects, the anti-CD3 scFv comprises the amino acid sequence of SEQ ID NO: 23. In exemplary aspects, the BsAb comprises the amino acid sequence of SEQ ID NO: 26. In exemplary instances, the second nucleotide sequence encodes an antigen-binding protein comprising, consisting essentially of, or consisting of a bispecific antibody (BsAb) that binds to CD3 and PSMA. In exemplary aspects, the BsAb comprises one, two, or three heavy chain CDR sequences of an anti-PSMA antigen binding protein, e.g., one, two, or three of SEQ ID NOs: 44-46 and/or one, two, or three light chain CDR sequences of an anti-PSMA antigen binding protein, e.g., one, two, or three of SEQ ID NOs: 47-49. In exemplary aspects, the BsAb comprises all six CDR sequences of an anti-PSMA antigen binding protein, e.g., all six of SEQ ID NOs: 44-49. In exemplary aspects, the BsAb comprises a heavy chain variable region sequence of SEQ ID NO: 37 or a light chain variable region sequence of SEQ ID NO: 39 or both SEQ ID NOs: 37 and 39. In exemplary aspects, the anti-PSMA antigen binding protein comprises the amino acid sequence of SEQ ID NO: 66. In exemplary instances, the second nucleotide sequence encodes an antigen-binding protein comprising, consisting essentially of, or consisting of a bispecific antibody (BsAb) that binds to CD3 and CD47. In exemplary aspects, the BsAb comprises a heavy chain variable region sequence of SEQ ID NO: 60 or a light chain variable region sequence of SEQ ID NO: 61 or both SEQ ID NOs: 60 and 61. In exemplary aspects, the anti-CD47 scFv comprises the amino acid sequence of SEQ ID NO: 59. In exemplary instances, the second nucleotide sequence encodes an antigen-binding protein comprising, consisting essentially of, or consisting of a bispecific antibody (BsAb) that binds to CD3 and CD19. In exemplary aspects, the BsAb comprises a heavy chain variable region sequence of SEQ ID NO: 62 or a light chain variable region sequence of SEQ ID NO: 63 or both SEQ ID NOs: 62 and 63.

In exemplary aspects, the second target is CD16a and the third target is GPC3, PSMA, CD47, or CD19. In exemplary instances, the second nucleotide sequence encodes an antigen-binding protein comprising, consisting essentially of, or consisting of BsAb that specifically binds to CD16a and GPC3, PSMA, CD47, or CD19. In exemplary aspects, the BsAb comprises a heavy chain variable region sequence of SEQ ID NO: 64 or a light chain variable region sequence of SEQ ID NO: 65 or both SEQ ID NOs: 64 and 65. In exemplary instances, the second nucleotide sequence encodes an antigen-binding protein comprising, consisting essentially of, or consisting of a bispecific antibody (BsAb) that binds to CD16a and GPC3. In exemplary aspects, the BsAb comprises one, two, or three heavy chain CDR sequences of an anti-GPC3 scFv, e.g., one, two, or three of SEQ ID NOs: 15-17 and/or one, two, or three light chain CDR sequences of an anti-GPC3 scFv, e.g., one, two, or three of SEQ ID NOs: 18-20. In exemplary aspects, the BsAb comprises all six CDR sequences of an anti-GPC3 scFv, e.g., all six of SEQ ID NOs: 15-20. In exemplary aspects, the BsAb comprises a heavy chain variable region sequence of SEQ ID NO: 21 or a light chain variable region sequence of SEQ ID NO: 22 or both SEQ ID NOs: 21 and 22. In exemplary aspects, the anti-CD16a scFv comprises the amino acid sequence of SEQ ID NO: 23. In exemplary aspects, the BsAb comprises the amino acid sequence of SEQ ID NO: 26. In exemplary instances, the second nucleotide sequence encodes an antigen-binding protein comprising, consisting essentially of, or consisting of a bispecific antibody (BsAb) that binds to CD16a and PSMA. In exemplary aspects, the BsAb comprises one, two, or three heavy chain CDR sequences of an anti-PSMA antigen binding protein, e.g., one, two, or three of SEQ ID NOs: 44-46 and/or one, two, or three light chain CDR sequences of an anti-PSMA antigen binding protein, e.g., one, two, or three of SEQ ID NOs: 47-49. In exemplary aspects, the BsAb comprises all six CDR sequences of an anti-PSMA antigen binding protein, e.g., all six of SEQ ID NOs: 44-49. In exemplary aspects, the BsAb comprises a heavy chain variable region sequence of SEQ ID NO: 37 or a light chain variable region sequence of SEQ ID NO: 39 or both SEQ ID NOs: 37 and 39. In exemplary aspects, the anti-PSMA antigen binding protein comprises the amino acid sequence of SEQ ID NO: 66. In exemplary instances, the second nucleotide sequence encodes an antigen-binding protein comprising, consisting essentially of, or consisting of a bispecific antibody (BsAb) that binds to CD16a and CD47. In exemplary aspects, the BsAb comprises a heavy chain variable region sequence of SEQ ID NO: 60 or a light chain variable region sequence of SEQ ID NO: 61 or both SEQ ID NOs: 60 and 61. In exemplary instances, the second nucleotide sequence encodes an antigen-binding protein comprising, consisting essentially of, or consisting of a bispecific antibody (BsAb) that binds to CD16a and CD19. In exemplary aspects, the BsAb comprises a heavy chain variable region sequence of SEQ ID NO: 62 or a light chain variable region sequence of SEQ ID NO: 63 or both SEQ ID NOs: 62 and 63.

Affinity and Avidity

The cell surface receptor encoded by the first nucleotide sequence and the antigen-binding protein encoded by the second nucleotide sequence bind to their target(s) in a non-covalent and reversible manner. In exemplary embodiments, the binding strength of the cell surface receptor or antigen-binding protein to its target may be described in terms of its affinity, a measure of the strength of interaction between the binding site of the cell surface receptor or antigen-binding protein and the target. In exemplary aspects, the antigen cell surface receptor or antigen-binding protein provided herein have high-affinity for their target(s) and thus will bind a greater amount of target(s) in a shorter period of time than low-affinity cell surface receptor or antigen-binding protein. In exemplary aspects, the cell surface receptor or antigen-binding protein has an equilibrium association constant, KA, which is at least 10⁵ mol⁻¹, at least 10⁶ mol⁻¹, at least 10⁷ mol⁻¹, at least 10⁸ mol⁻¹, at least 10⁹ mol⁻¹, or at least 10¹⁰ mol⁻¹. As understood by the artisan of ordinary skill, KA can be influenced by factors including pH, temperature and buffer composition.

In exemplary embodiments, the binding strength of the cell surface receptor or antigen-binding protein to their target(s) may be described in terms of its sensitivity. KD is the equilibrium dissociation constant, a ratio of k_{off}/kₒₙ, between the cell surface receptor or antigen-binding protein and their target(s). KD and KA are inversely related. The KD value relates to the concentration of the cell surface receptor or antigen-binding protein (the amount of cell surface receptor or antigen-binding protein needed for a particular experiment) and so the lower the KD value (lower concentration) the higher the affinity of the cell surface receptor or antigen-binding protein. In exemplary aspects, the binding strength of the cell surface receptor or antigen-binding protein to their target(s) may be described in terms of KD. In exemplary aspects, the KD of the cell surface receptor or antigen-binding protein is about 10⁻¹, about 10⁻², about 10⁻³, about 10⁻⁴, about 10⁻⁵, about 10⁻⁶, or less. In exemplary aspects, the KD of the cell surface receptor or antigen-binding protein provided herein is micromolar, nanomolar, picomolar or femtomolar. In exemplary aspects, the KD of the cell surface receptor or antigen-binding protein is within a range of about 10⁻⁴ to 10⁻⁶ or 10⁻⁷ to 10⁻⁹ or 10⁻¹⁰ to 10⁻¹² or 10⁻¹³ to 10⁻¹⁵. In exemplary aspects, the KD of the cell surface receptor or antigen-binding protein is within a range of about 1.0×10⁻¹² M to about 1.0×10⁻⁸ M. In exemplary aspects, the KD of the cell surface receptor or antigen-binding protein is within a range of about 1.0×10⁻¹¹ M to about 1.0×10⁻⁹ M.

Avidity gives a measure of the overall strength of an antigen-binding protein-antigen complex. It is dependent on three major parameters: affinity of the antigen-binding protein for the epitope, valency of both the cell surface receptor or antigen-binding protein and their target(s), and structural arrangement of the parts that interact. The greater the valency of the cell surface receptor or antigen-binding protein (number of antigen binding sites), the greater the amount of target it can bind. In exemplary aspects, the cell surface receptor or antigen-binding protein has a strong avidity for their target(s). In exemplary aspects, the cell surface receptor or antigen-binding protein is multivalent. In exemplary aspects, the cell surface receptor or antigen-binding protein is bivalent.

Compositions, Pharmaceutical Compositions and Formulations

The present disclosure also provides related compositions. In exemplary embodiments, the composition comprises (i) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. In exemplary aspects, expression of the second nucleotide sequence is activated only when the first target binds to the cell surface receptor. In exemplary aspects, the first nucleotide sequence is operably linked to a constitutive promoter, e.g., in accordance with the teachings herein. In exemplary aspects, the constitutive promoter is a human promoter, e.g., human elongation factor-1 alpha (EF-1α) or a functional variant thereof. In exemplary aspects, the human promoter comprises a nucleotide sequence of SEQ ID NO: 1. In some instances, the inducible promoter operably linked to the second nucleotide sequence comprises a binding site for a protein expressed upon binding of the first target to the cell surface receptor. In some aspects, the protein is a transcription factor expressed in activated T-cells. In exemplary aspects, the transcription factor is an Nuclear Factor of Activated T-Cells (NFAT), AP-1, or NF-κB or the transcription factor is IRF4, Rel, T-bet, Blimp-1, BATF, or Nur77. In exemplary aspects, the nucleic acid comprising the first nucleotide sequence also comprises the second nucleotide sequence. In exemplary aspects, the composition comprises a vector comprising both the first nucleotide sequence and the second nucleotide sequence. In alternative aspects, the nucleic acid comprising the first nucleotide sequence is distinct from the nucleic acid comprising the second nucleotide sequence. For example, the composition comprises a first vector and a second vector, wherein the first vector comprises the first nucleotide sequence and the second vector comprises the second nucleotide sequence.

In exemplary embodiments, the compositions comprises a cell, or a population thereof, comprising (a) a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor.

The compositions provided herein may comprise agents which enhance the chemico-physico features of the cells or nucleic acid(s), e.g., via stabilizing the cells or nucleic acid(s) at certain temperatures, e.g., room temperature, increasing shelf life, reducing degradation, e.g., oxidation protease mediated degradation, increasing half-life of the cells or nucleic acid(s), etc. In exemplary embodiments, the composition is a pharmaceutical composition comprising the cell, or population thereof, or nucleic acid(s) and a pharmaceutically acceptable carrier, diluent, or excipient. In this regard, the present disclosure further provides pharmaceutical compositions comprising the cell, or a population thereof, and/or nucleic acid(s), that are intended for administration to a subject, e.g., a mammal.

In some embodiments, the cell, or a population thereof, and/or nucleic acid(s), is present in the pharmaceutical composition at a purity level suitable for administration to a subject. In some embodiments, the cell, or a population thereof, and/or nucleic acid(s), has a purity level of at least about 90%, about 91%, about 92%, about 93%, about 94%, about 95%, about 96%, about 97%, about 98% or about 99%, and a pharmaceutically acceptable diluent, carrier or excipient.

In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to subjects at the dosages and concentrations employed. In specific embodiments, pharmaceutical compositions comprising the cell, or a population thereof, and/or nucleic acid(s), and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In exemplary embodiments, the pH of the pharmaceutical composition is between 5.5 and 7.5.

Routes of Administration

With regard to the present disclosure, the pharmaceutical composition comprising the cell, population of cells, or nucleic acid(s), can be administered to the subject via any suitable route of administration. For example, the pharmaceutical composition can be administered to a subject via parenteral, nasal, oral, pulmonary, topical, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The pharmaceutical composition can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-I53-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the composition of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Dosages

The compositions of the disclosure are believed to be useful in methods of killing a diseased or infected cell, as well as other methods, as further described herein, including methods of treating or preventing cancer. For purposes of the disclosure, the amount or dose of the composition administered should be sufficient to effect, e.g., a therapeutic or prophylactic response, in the subject or animal over a reasonable time frame. For example, the dose of the composition should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the composition of the present disclosure to a mammal among a set of mammals, each set of which is given a different dose of the composition, could be used to determine a starting dose to be administered to a mammal. The extent to which cancer is treated upon administration of a certain dose can be represented by, for example, the cytotoxicity of the active agent or the extent of tumor regression achieved with the active agent in a mouse xenograft model. Methods of measuring cytotoxicity of cells of the present disclosure and methods of assaying tumor regression are known in the art.

The dose of the composition of the present disclosure also will be determined by the existence, nature and extent of any adverse side effects that might accompany the administration of a particular composition of the present disclosure. Typically, the attending physician will decide the dosage of the composition of the present disclosure with which to treat each individual patient, taking into consideration a variety of factors, such as age, body weight, general health, diet, sex, composition of the present disclosure to be administered, route of administration, and the severity of the condition being treated.

Methods of Use

The cells of the present disclosure are believed to be useful for killing a diseased or infected cell. Thus, the present disclosure further provides a method of killing a diseased or infected cell. In exemplary embodiments, the method comprises contacting the diseased or infected cell with the cells of the present disclosure, the composition comprising the nucleic acid(s) of the present disclosure, the therapeutic cells produced by the method of manufacture of the present disclosure, or a combination thereof. In exemplary aspects, the contacting step is carried out in vivo. For example, the cells, e.g., therapeutic cells, of the present disclosure comprise a cell surface receptor that binds to an antigen of the diseased or infected cell and upon delivery to a subject comprising the diseased or infected cell, the cells, e.g., therapeutic cells, of the present disclosure cause specific lysis of the diseased or infected cell. In alternative or additional aspects, the contacting is carried out in vitro. For example, the composition comprising the nucleic acid(s) of the present invention are introduced into cells (e.g., T cells or NK cells) obtained from a subject comprising the diseased or infected cell, and upon delivery of the cells containing the nucleic acid(s) to the subject, the cells cause specific lysis of the diseased or infected cell.

Without being bound to a particular theory, the killing of diseased or infected cells leads to treatment of the disease or infection. Accordingly, the invention provides methods of treating a subject with a disease. In exemplary aspects, the method comprises administering to the subject the pharmaceutical composition of the present invention in an amount effective to treat the disease, wherein the first target and third target are expressed by a cell of the disease. The disease may be cancer or a tumor or an infectious disease. In exemplary aspects, the disease is an infection by a pathogen. In exemplary aspects, the disease is an infectious disease listed in Table 2. In exemplary aspects, the pathogen is a virus, such as one listed in Table 2. In exemplary aspects, the cancer is one which is described herein. For example, the disease is a cancer optionally selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer and thyroid cancer.

Also provided herein is a method of treating a viral infection in a subject, comprising administering to the subject the pharmaceutical composition of the present disclosure in an amount effective to treat the viral infection, wherein the first target and/or third target are viral antigens. In exemplary aspects, the viral infection is caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), Human immunodeficiency virus (HIV), and Hepatitis C Virus (HCV).

With regard to any of the methods of the present disclosure, the cells may be autologous to the subject being treated. Accordingly, in exemplary aspects, the method of treating a subject with a tumor or cancer or a viral infection comprises (a) obtaining immune cells from a subject, (b) contacting the cells with a composition comprising the nucleic acid(s) of the present disclosure, and (c) administering the cells to the subject in an amount effective to treat the tumor or cancer or viral infection.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way of increasing the survival of the subject.

Subjects

In some embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

Methods of Manufacture

The present disclosure further provides a method of making a cell as presently disclosed. The cell may be considered a therapeutic cell if the first and third targets are antigens of a diseased or infected cell. In exemplary aspects, the method comprises contacting a cell with a composition comprising (i) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (ii) a nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor. The composition comprising the nucleic acid may be any of those described herein. In exemplary aspects, the cell which is contacted with the composition is an immune cell. In exemplary aspects, the cell is obtained from a human. In some aspects, the method comprises obtaining immune cells from a human then contacting the cells with the expression vector system. In exemplary aspects, the method comprises culturing the cells for a time period sufficient to expand the cells to a population of at least 10⁶ cells. In exemplary aspects, the cells are expanded to a population of at least 10⁷, 10⁸, 10⁹, 10¹⁰, 10¹¹, 10¹² or more cells.

Methods of delivering nucleic acids for expression in cells are known in the art and include for example, lipid delivery using cationic lipids or other chemical methods (e.g., calcium phosphate precipitation, DEAE-dextran, polybrene), electroporation, or viral delivery. See, e.g., Sambrook and Russell, Molecular Cloning: A Laboratory Manual, 3^{rd} ed. Cold Spring Harbor Press, Cold Spring Harbor, N.Y. (2001), Nayerossadat et al., Adv Biomed Res 1: 27 (2012); and Hesier, William (ed.), Gene Delivery to Mammalian Cells, Vol 1., Non-viral Gene Transfer Techniques, Methods in Molecular Biology, Humana Press, (2004).

Kits

In exemplary aspects, the cells, therapeutic cells, or compositions comprising one or more nucleic acid(s) are provided as a kit. Accordingly, the present disclosure provides a kit comprising the cells of the present disclosure, the composition of the present disclosure, the therapeutic cells produced by the method of manufacture of the present disclosure, or a combination thereof, and a device for administration of the cells, composition, or therapeutic cells.

In exemplary aspects, the kit comprises the cell(s) and/or nucleic acid(s) as a unit dose. For purposes herein “unit dose” refers to a discrete amount dispersed in a suitable carrier. In exemplary aspects, the unit dose is the amount sufficient to provide a subject with a desired effect, e.g., killing of a diseased or infected cell, treatment of cancer or an infection. In exemplary aspects, the unit dose is at least or about 10⁶, 10⁷, 10⁸, 10⁹, 10¹⁰, 10¹¹, 10¹² cells or the present disclosure. In exemplary aspects, the kit comprises several unit doses, e.g., a week or month supply of unit doses, optionally, each of which is individually packaged or otherwise separated from other unit doses. In exemplary aspects, the cells are cryopreserved and the kit comprises reagents or instructions for thawing the cells. In some embodiments, the components of the kit/unit dose are packaged with instructions for administration to a patient. In some embodiments, the kit comprises one or more devices for administration to a patient, e.g., a needle and syringe, and the like. In some aspects, the cell(s) and/or nucleic acid(s) is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein.

Articles of Manufacture

In exemplary aspects, the cells, therapeutic cells, or compositions comprising one or more nucleic acid(s) are provided as an article of manufacture comprising the the cells, therapeutic cells, or compositions, or a combination thereof, housed in a container. The container may be a dish, plate, vial, flask, bag, tube, or other suitable container. In exemplary aspects, the article of manufacture comprises the cell(s) and/or nucleic acid(s) as a unit dose.

### Exemplary Embodiments

In exemplary embodiments, the presently disclosed cell is an immune cell comprising: (a) a nucleic acid encoding a chimeric antigen receptor (CAR) which binds to a first antigen; and (b) a nucleic acid encoding an antibody which binds to a second antigen and third antigen, wherein the first antigen is different from the third antigen, wherein the expression of the nucleic acid encoding a bispecific antibody is activated upon binding of the first antigen to the cell surface receptor, wherein the first antigen and/or third antigen is a peptide of an intracellular protein bound to an MHC molecule.

In exemplary embodiments, the presently disclosed cell is a T-cell comprising (a) a first nucleic acid comprising a first nucleotide sequence encoding a chimeric antigen receptor (CAR) comprising an extracellular domain (ECD) which binds to a first target, a transmembrane domain (TMD), and an intracellular domain (ICD) comprising at least a portion of a T-cell signaling molecule, and (b) a second nucleic acid comprising a second nucleotide sequence encoding a bispecific antibody which binds to a second target and a third target, wherein the second nucleotide sequence is operably linked to an inducible promoter, wherein expression of the second nucleotide sequence is activated upon binding of the first target to the CAR, wherein each of the first target and third target is an antigen expressed by a solid tumor, wherein the first target is different from the third target, and wherein the second target is expressed on the surface of a T-cell or a natural killer cell.

In additional exemplary embodiments, the presently disclosed cell is a T-cell comprising: (a) a first nucleic acid comprising a first nucleotide sequence encoding a chimeric antigen receptor (CAR) comprising an extracellular domain (ECD) which binds to an alpha fetoprotein (AFP) peptide bound to an MHC molecule, a transmembrane domain (TMD), and an intracellular domain (ICD) comprising at least a portion of CD28 and CD3, and (b) a second nucleic acid comprising a second nucleotide sequence encoding a bispecific antibody which binds to GPC3 and CD3, wherein the second nucleotide sequence is operably linked to an inducible NFAT promoter, wherein expression of the second nucleotide sequence is activated upon binding of the AFP peptide bound to the MHC molecule to the CAR.

The following examples are given merely to illustrate the present invention and not in any way to limit its scope.

### EXAMPLES

### Example 1

Construction and Characterization of T Cells Transduced with Vectors Encoding Anti-AFP CAR and Anti-GPC3/Anti-CD3 BsAb

Generation of Vectors Expressing Cell Surface Receptors and Antibodies

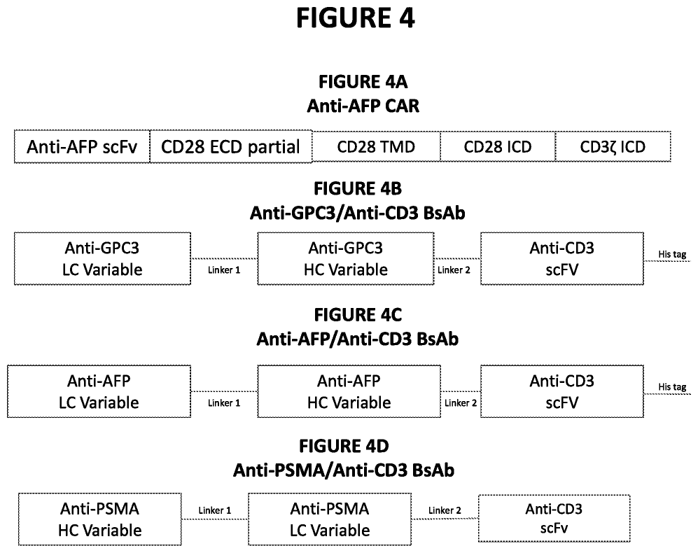

A lentiviral chimeric antigen receptor (CAR) expression vector comprising a nucleotide sequence encoding a semi-synthetic CAR with specificity for an AFP peptide (amino acids 158-166 of human AFP (SEQ ID NO: 67)) complexed with a human leukocyte antigen (HLA) was constructed. The encoded CAR comprised an scFV which binds to the AFP peptide complexed with HLA-A*02:01. An anti-AFP158/HLA-A*02:01 scFv-expressing CAR (or anti-AFP CAR) was essentially that described in Liu et al., Clin Cancer Res 23(2): 478-488 (2017). Briefly, an anti-AFP158/HLA-A*02:01 scFv sequence was grafted onto a second generation CAR (Mackall, C. L. et al., Nat. Rev. Clin. Oncol. 11(12):693-703, 2014) comprising CD28 signaling domain and CD3ζ (also known as TCRζ) engineered in cis to provide intracellular T cell stimulation signals and to activate T cells. A schematic of the anti-AFP158/HLA-A*02:01 CAR construct is provided in FIG. 4A. Amino acid sequences of the heavy chain and light chain variable domains of the anti-AFP scFv are shown in Table 3 below and in FIG. 4A. A nucleotide sequence encoding the anti-AFP CAR was cloned into a pCDH lentiviral vector (Systems Biosciences) under the control of the EF1-alpha promoter (SEQ ID NO: 1), a constitutive promoter.

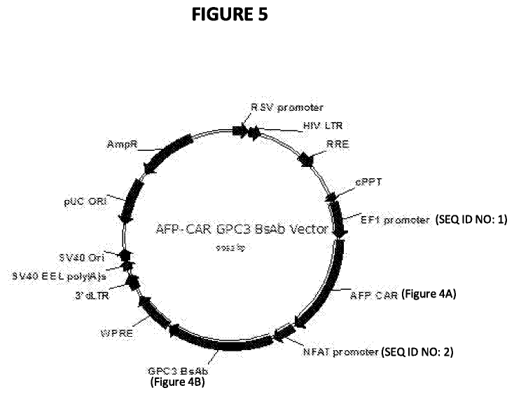

A lentiviral vector comprising a nucleic acid encoding an anti-GPC3/anti-CD3 bispecific antibody (BsAb) was generated. The nucleic acid comprised a nucleotide sequence encoding the anti-GPC3/anti-CD3 BsAb as shown in FIG. 4B. Linkers located between the variable regions and the scFV are shown. The nucleic acid was cloned into the lentiviral vector under the control of an NFAT-derived promoter (SEQ ID NO: 2) comprising six NFAT response elements (SEQ ID NO: 3) and a minimal TA promoter (SEQ ID NO: 14). Such a promoter was inducible by antigen recognition and activation of the T-cell through the anti-AFP CAR molecule. In particular, the promoter was activated upon antigen binding to the AFP CAR. The lentiviral vector was constructed so that it encoded both the anti-GPC3/anti-CD3 BsAb described above and the above-described anti-AFP CAR-encoding nucleotide sequence under the constitutive promoter. A schematic of the lentiviral vector encoding both the anti-GPC3/anti-CD3 BsAb and the anti-AFP CAR is provided in FIG. 5. The amino acid sequences of each part of the anti-AFP CAR and the BsAb diagrammed in FIGS. 4A and 4B are provided by Table 3 and the sequence listing submitted herewith.

TABLE 3

| | | SEQ |
|---|---|---|
| Product | Description | ID NO: |
| Anti-AFP CAR | Signal Peptide | 30 |
| | Anti-AFP scFv | 31 |
| | CD28 ECD partial | 33 |
| | CD28 TMD | 34 |
| | CD28 ICD | 35 |
| | CD3ζ ICD | 36 |
| Anti-GPC3/Anti- | Anti-GPC3 light chain (LC) | 22 |
| CD3 BsAb | variable region | |
| | Linker 1 | 27 |
| | GPC3 heavy chain (HC) | 21 |
| | variable region | |
| | Linker 2 | 25 |
| | Anti-CD3 scFv | 24 |

Generation of Anti-AFP-CAR T Cells and T Cells Expressing Anti-AFP-CAR and Anti-GPC3/Anti-CD3 Bispecific Antibody (BsAb)

T cells were prepared from PBMC-enriched whole blood using EasySep™ Human T Cell Isolation Kit (StemCell Technologies). The kit allows for selection of T-cells through the depletion of cells expressing CD14, CD16, CD19, CD20, CD36, CD56, CD66b, CD123, and/or glycophorin A. The selected human T-cells were cultured in RPMI1640 supplemented with 10% FBS, activated and expanded with, for example, CD3/CD28 Dynabeads® (Thermo Fisher Scientific) in the presence of interleukin-2 (IL-2) at 100 U/ml for one day, and used for transduction. Concentrated lentiviruses encoding only the anti-AFP CAR or both the anti-AFP CAR and the anti-GPC3/anti-CD3 BsAb were applied to the T-cells for 96 hours in 6-well plates coated with Retronectin (Takara Bio USA). Transduced T cells were then expanded in the presence of either 100 U/mL IL-2 (Sigma) or 10 ng/mL IL-7 and 5 ng/mL IL-15 (Peprotech) for 8-12 days. Transduction efficiencies were assessed by flow cytometry for measurements of the % of CAR-positive cells by staining with Streptavidin-PE-conjugated AFP₁₅₈/HLA-A*02:01 tetramer.

### Example 2

In Vitro Cytotoxicity of Anti-AFP-CAR T Cells and T Cells Expressing Anti-AFP-CAR and Anti-GPC3/Anti-CD3 BsAb

This example demonstrates the ability of the cells of the present disclosure to kill cancer cells.

Cell Samples and Cell Lines

Primary T cells used in the Examples are peripheral blood lymphocytes isolated from healthy donors. T cells expressing anti-AFP-CAR and T cells expressing anti-AFP-CAR and anti-GPC3/anti-CD3 BsAb were generated as described in Example 1. CD3⁺ T cells were prepared from PBMC-enriched whole blood using EasySep Human T Cell Isolation Kit (StemCell Technologies) which depletes CD14, CD16, CD19, CD20, CD36, CD56, CD66b, CD123, glycophorin A expressing cells.

T cells expressing either CAR alone or CAR and BsAB were assayed for target cell killing. The target cells used in the assay included cells of the cell lines described in Table 4:

TABLE 4

| | HLA-A02L01 | AFP | GPC3 | |
|---|---|---|---|---|
| | expres- | expres- | expres- | Reference or |
| | sion | sion | sion | ATCC no. |
| Liver cancer | + | + | + | Lopez-Terrada |
| cell line | | | | et al., |
| HepG2 | | | | Hum Pathol |
| | | | | 40: 1512-1515 |
| | | | | (2009); |
| | | | | ATCC |
| | | | | HB-8065 |
| Liver cell line | + | − | − | ATCC |
| SK-HEP1 | | | | HTB-52 |
| Liver Cancer | + | + | − | USSN |
| Cell line | | | | 62/490,586 |
| HepG2 GPC3 | | | | |
| knockout | | | | |
| (HepG2- | | | | |
| GPC3 ko) | | | | |
| SK-HEP1-GPC3 | + | − | + | USSN |
| knock in | | | | 62/490,586 |

The full-length GPC3 sequence is provided as SEQ ID NO: 68.

All cell lines were cultured in RPMI 1640 or DMEM supplemented with 10% FBS and 2 mM glutamine at 37° C./5% CO₂.

T-cells were matched to the same anti-AFP CAR-positive percentages by mixing with mock T-cells (primary T cells undergone the same transduction procedure without a vector). T cells, expressing both anti-AFP CAR (abbreviated as “AFP CAR”) and anti-GPC3/anti-CD3 BsAb (abbreviated as “GPC3-CD3 BsAb”) or just anti-AFP CAR, and each of the four different target cell lines were mixed at an effector-to-target ratio (number of anti-AFP CAR-positive T cells to target cells) of 5:1. Specific T-cell lysis was measured after a 16-hr incubation using the Cytox 96 Non-radioactive LDH Cytotoxicity Assay (Promega).

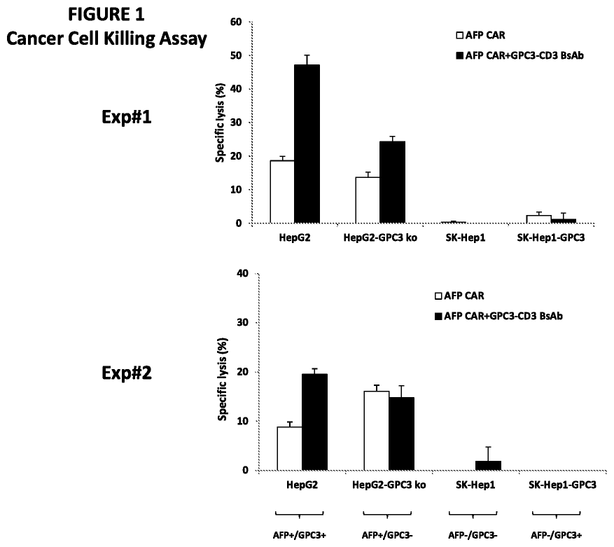

Representative results of two cell killing assays are shown in FIG. 1. As shown in FIG. 1, the greatest amount of specific lysis of target cells was achieved by T cells expressing both the anti-AFP CAR and the anti-GPC3/anti-CD3 BsAb. Specific lysis also occurred in cells expressing just the anti-AFP CAR, but to a lesser extent.

These data support that cells expressing both the anti-AFP CAR and the anti-GPC3/anti-CD3 BsAb provide potent cancer cell killing.

### Example 3

In Vitro Cytokine Release by Anti-AFP-CAR T Cells and T Cells Expressing Anti-AFP-CAR and Anti-GPC3/Anti-CD3 BsAb

This example demonstrates the ability of the therapeutic cells of the present disclosure to release cytokines as an indicator of T cell activity.

To characterize and compare the ability of cells expressing both the AFP CAR and BsAb to release cytokines, in vitro cytokine release assays were carried out using the T cells expressing both AFP CAR and BsAb and T cells expressing just AFP CAR. T-cells were matched at 50% AFP CAR-positive by mixing with mock T-cells. 100,000 HepG2 and HepG2 GPC3 k.o. cells were prepared and incubated with 500,000 T cells expressing both the AFP CAR and GPC3-CD3_BsAb or just AFP CAR. Release of IL-2, TNFα, GMCSF, and IFNγ was measured after 16 hr incubation with Luminex beads using a Bio-Plex Pro kit (BioRad).

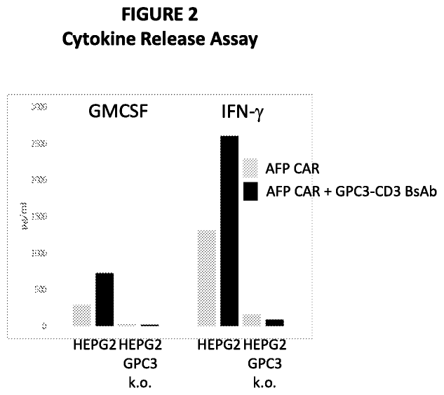

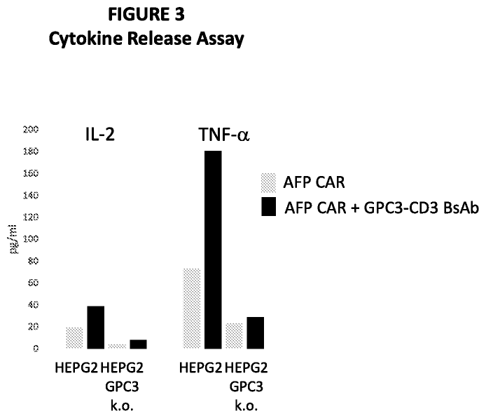

The results of the cytokine release assays are shown in FIGS. 2 and 3. Significant levels of IFNγ and TNFα were released by T cells expressing both the AFP CAR and BsAb when stimulated by HepG2 cells, which express both AFP/MHC and GPC3, indicating higher T cell activities. The highest amounts of secreted IL-2 and GMCSF were also achieved by T cells expressing both the AFP CAR and BsAb when stimulated by HepG2 cells. The cytokines were also released by T cells expressing AFP CAR only, albeit to a much lesser extent.

### Example 4

This example demonstrates an exemplary method of testing mouse xenograft models of liver cancer with cells of the present disclosure.

Animal experiments are conducted at a research contract laboratory. Female NSG mice aged 6-8 weeks are used. For subcutaneous tumor models, cells are mixed with 50% Matrigel prior to implantation and tumors are measured by calipers. For Hep G2 tumor models, 2.5×10⁶ cells are implanted subcutaneously (s.c.) over the right flank of each mouse; for SK-HEP-1-MG models, 5×10⁶ cells are implanted. When tumors reach approximately 100 mm³, mice are randomized to treatment groups, with 6-8 mice per group: 1) intratumoral (i.t.) injection of untransduced, donor-matched T cells (mock), 2) i.t. injection of T cells transduced with AFP158 CAR only (described in Example 2), and 3) i.t. injection of T cells transduced with AFP158 CAR and inducible GPC3/CD3 BsAb (described in Example 2). Treatments are administered at a dose range of between 2 million and 8 million CAR-positive cells per mouse. The animals in each group are monitored for tumor volume, adverse response, body weight, and general health condition (eating, walking, daily activities).

### Example 5

This example demonstrates the construction of T cells expressing an AFP CAR and an AFP/CD3 BsAb.

The prior examples demonstrate the superior cell killing and cytokine release activities of T cells constitutively expressing an AFP CAR and inducibly expressing a GPC3/CD3 BsAb (expression induced by AFP CAR binding to the appropriate antigen), relative to T cells expressing AFP CAR alone (i.e., without expression of GPC3/CD3 BsAb). To evaluate whether it is important to have the CAR recognize a different target from the target recognized by the BsAb, a lentiviral vector encoding an AFP/CD3 BsAb is constructed as essentially described in Example 1, except that the sequences of the GPC3 heavy and light chain variable regions (SEQ ID NOs: 21 and 22, respectively) are replaced with the sequences of the AFP heavy and light chain variable regions sequences (SEQ ID NOs: 28 and 29, respectively as depicted in FIG. 4C). The amino acid sequences of each part of the anti-AFP/Anti-CD3 BsAb diagrammed in FIG. 4C are provided by Table 5 and the sequence listing submitted herewith.

TABLE 5

| | | SEQ |
|---|---|---|
| Product | Description | ID NO: |
| Anti-AFP CAR | Signal Peptide | 30 |
| | Anti-AFP scFv | 31 |
| | CD28 ECD partial | 33 |
| | CD28 TMD | 34 |
| | CD28 ICD | 35 |
| | CD3ζ ICD | 36 |
| Anti-AFP/Anti- | Anti-AFP LC variable region | 29 |
| CD3 BsAb | Linker 1 | 27 |
| | Anti-AFP HC variable region | 28 |
| | Linker 2 | 25 |
| | Anti-CD3 scFv | 24 |

Lentiviral vectors comprising a nucleotide sequence encoding the AFP CAR (operably linked to a constitutive promoter) and a nucleotide sequence encoding the AFP/CD3 BsAb (operably linked to an inducible promoter) are expressed in CD3+ T cells selected from PBMCs isolated from whole blood.

Transduction efficiencies of the AFP CAR and the AFP/CD3 BsAb are assayed by flow cytometry as described in Example 1 and in vitro cytotoxicity against target cells are tested as essentially described in Example 2. Cytokine release abilities are furthermore assayed following the procedures described in Example 3. In vivo activities are tested by the methodology described in Example 4. The target cell killing and cytokine release activities of the T cells expressing AFP CAR and AFP/CD3 BsAb are compared against those activities of T cells expressing AFP CAR and GPC3/CD3 BsAb. It is expected that the T cells expressing AFP CAR and GPC3/CD3 BsAb exhibit activities that are about the same if not superior to the activities displayed by the T cells expressing AFP CAR and AFP BsAb. Development of the T cells expressing AFP CAR and GPC3/CD3 BsAb to two different targets are expected to provide higher tumor regression rates.

### Example 6

This example demonstrates the construction of another exemplary cell of the present disclosure.

A lentiviral vector encoding a PSMA/CD3 BsAb is constructed as essentially described in Example 1, except that the sequence of the GPC3 heavy and light chain variable regions are replaced with the variable region sequences of a PSMA antigen binding protein (see FIG. 7 and US Patent Publication No. 2011293619A). The amino acid sequences of each part of the anti-PSMA/Anti-CD3 BsAb diagrammed in FIG. 4D are provided by Table 6 and the sequence listing submitted herewith.

TABLE 6

| | | SEQ |
|---|---|---|
| Product | Description | ID NO: |
| Anti-PSMA/Anti- | Anti-PSMA HC variable | 37 |
| CD3 BsAb | region | |
| | Linker 1 | 38 |
| | Anti-PSMA LC variable region | 39 |
| | Linker 2 | 40 |
| | Anti-CD3 scFv | 41-43 |

The lentiviral vector is engineered to further comprise a nucleotide sequence encoding a CAR that is specific for a PSA peptide (KLQCVDLHV (SEQ ID NO: 69) complexed to HLA-A*02:01 (PSA/HLA CAR) operably linked to a constitutive promoter. The sequence can be any one of those described in International Patent Application Publication No. WO2017015634 (see Table 9 describing Lv and Hv sequences of clones 3, 10, 20, and 26) which is incorporated by reference in its entirety. The nucleotide sequence encoding the PSMA/CD3 BsAb is operably linked to an inducible promoter. CD3+ T cells selected from PBMCs isolated from whole blood are transduced as essentially described in Example 1 but using the new vectors.

Transduction efficiencies of the PSA/HLA CAR and the PSMA/CD3 BsAb are assayed by flow cytometry as described in Example 1 and in vitro cytotoxicity against target cells are tested as essentially described in Example 2. The target cells used in the cytotoxicity assays include prostate cancer cell lines, including, but not limited to, LNCaP (ATCC CRL-1740; HLA-A2⁺, PSA⁺) is double positive for HLA-A2 and PSA, while MDA PCa 2b (ATCC CRL-2422; HLA-A2⁻, PSA⁺), VCaP (ATCC CRL-2876; HLA-A2⁻, PSA⁻), 22Rv1 (ATCC CRL-2505; HLA-A2⁻, PSA⁻), and PC3 (ATCC CRL-1435; HLA-A2⁻, PSA⁻). Breast cancer cell line MDA-MB-231 (ATCC HTB-26; HLA-A2⁺, PSA⁻) and liver cancer cell line SK-Hep1 (ATCC HTB-52; HLA-A2⁺, PSA⁻) are used as negative controls. MDA-MB-231-MiniG (HLA-A2⁺, PSA⁺) and SK-HEP-1-MiniG (HLA-A2⁺, PSA⁺) are used as positive controls. More particularly, MDA-MB-231 and/or SK-HEP-1 cells are transduced with a PSA peptide expressing a minigene cassette, which results in a high level of cell surface expression of PSA peptide/HLA-A*02:01 complex. Cytokine release abilities are furthermore assayed following the procedures described in Example 3.

### Example 7

This example demonstrates an exemplary method of testing mouse xenograft models of prostate cancer with cells of the present disclosure.

HLA-A02⁺/PSA⁺ sub-cutaneous (s.c.) tumor xenograft models are generated in NSG mice (no functional T-, B-, NK-cells) by transplanting MDA-MB-231 MiniG (HLA-A2⁺, PSA⁺) cells subcutaneously (s.c.) over the right flank. Animals are randomized when average s.c. tumor volume reaches approximately 100 mm³. 24 hours prior to CAR-T administration, animals are treated (via intraperitoneal route) with 60 mg/kg cyclophosphamide. Mice are divided into 4 groups (n=6-8 mice/group) that receive one of the following: (1) no treatment; (2) 10⁷ mock transduced CAR T cells; 3) treatment with T cells transduced with PSA CAR only (described in Example 6), and 4) treatment with T cells transduced with PSA CAR and inducible PSMA/CD3 BsAb (described in Example 6)., 1×/week for 4 weeks. The animals in each group are monitored for tumor volume, adverse response, human cytokine profile, histopathology of tumor for human CD3⁺ cells in tumor and organs for CAR T cell infiltration, serum PSA, body weight and general health condition (eating, walking, daily activities).

SEQUENCE LISTING

| | | SEQ |
|---|---|---|
| Sequence | | ID |
| Description | Sequence | NO: |
| EF1-alpha | GGATCTGCGATCGCTCCGGTGCCCGTCAGTGGGCAGAGCGCACATCGCCCACAGTCCCCGAGAAGTTGGG | 1 |
| promoter | GGGAGGGGTCGGCAATTGAACGGGTGCCTAGAGAAGGTGGCGCGGGGTAAACTGGGAAAGTGATGTCG | |
| | TGTACTGGCTCCGCCTTTTTCCCGAGGGTGGGGGAGAACCGTATATAAGTGCAGTAGTCGCCGTGAACGT | |
| | TCTTTTTCGCAACGGGTTTGCCGCCAGAACACAGCTGAAGCTTCGAGGGGCTCGCATCTCTCCTTCACGCG | |
| | CCCGCCGCCCTACCTGAGGCCGCCATCCACGCCGGTTGAGTCGCGTTCTGCCGCCTCCCGCCTGTGGTGCC | |
| | TCCTGAACTGCGTCCGCCGTCTAGGTAAGTTTAAAGCTCAGGTCGAGACCGGGCCTTTGTCCGGCGCTCCC | |
| | TTGGAGCCTACCTAGACTCAGCCGGCTCTCCACGCTTTGCCTGACCCTGCTTGCTCAACTCTACGTCTTTGTT | |
| | TCGTTTTCTGTTCTGCGCCGTTACAGATCCAAGCTGTGACCGGCGCCTAC | |
| NFAT-derived | GGAGGAAAAACTGTTTCATACAGAAGGCGTGGAGGAAAAACTGTTTCATACAGAAGGCGTGGAGGAAAA | 2 |
| promoter | ACTGTTTCATACAGAAGGCGTGGAGGAAAAACTGTTTCATACAGAAGGCGTGGAGGAAAAACTGTTTCAT | |
| | ACAGAAGGCGTGGAGGAAAAACTGTTTCATACAGAAGGCGTCTCGAGGCCGCCCCGACTGCATCTGCGTG | |
| | TTCCAATTCGCCAATGACAAGACGCTGGGCGGGGTTTGTGTCATCATAGAACTAAAGACATGCAAATATAT | |
| | TTCTTCCGGGGACACCGCCAGCAAACGCGAGCAACGGGCCACGGGGATGAAGCAG | |
| six NFAT | GGAGGAAAAACTGTTTCATACAGAAGGCGTGGAGGAAAAACTGTTTCATACAGAAGGCGTGGAGGAAAA | 3 |
| response | ACTGTTTCATACAGAAGGCGTGGAGGAAAAACTGTTTCATACAGAAGGCGTGGAGGAAAAACTGTTTCAT | |
| elements | ACAGAAGGCGTGGAGGAAAAACTGTTTCATACAGAAGGCGT | |
| NFAT consensus | GGAAA | 4 |
| binding site | | |
| NFAT/AP-1 | GGAAAAACTGTTTCA | 5 |
| consensus | | |
| sequence | | |
| NF-κB consensus | GGGACTTTCC | 6 |
| sequence | | |
| ISRE | AGTTTCNNTTTC | 7 |
| | AGTTTCACTTCT | 8 |
| | AGTTTCCCGTTC | 9 |
| | AGTTTCTCTTTC | 10 |
| | ATTCTCTCCTCC | 11 |
| | AGTTTCTTTTCT | 12 |
| | Gobin et al. Immunol 163: 1428-1434 (1999) | |
| CD47 ECD | L LFNKTKSVEF TFCNDTVVIP CFVTNMEAQN TTEVYVKWKF KGRDIYTFDG ALNKSTVPTD FSSAKIEVSQ | 13 |
| | LLKGDASLKM DKSDAVSHTG NYTCEVTELT REGETIIELK YRVVSWFSPN EN | |
| minimal TA | GCCGCCCCGACTGCATCTGCGTGTTCCAATTCGCCAATGACAAGACGCTGGGCGGGGTTTGTGTCATCATA | 14 |
| promoter | GAACTAAAGACATGCAAATATATTTCTTCCGGGGACACCGCCAGCAAACGCGAGCAACGGGCCACGGGGA | |
| | TGAAGCAG | |
| anti-GPC3 HC- | GFTFSSYA | 15 |
| CDR1 | | |
| anti-GPC3 HC- | IYSGGSST | 16 |
| CDR2 | | |
| anti-GPC3 HC- | ARTSYLNHGDY | 17 |
| CDR3 | | |
| anti-GPC3 LC- | RSNIGSDY | 18 |
| CDR1 | | |
| anti-GPC3 LC- | GDN | 19 |
| CDR2 | | |
| anti-GPC3 LC- | GTWDYTLNGVV | 20 |
| CDR3 | | |
| IgVH domain of | QVQLVESGGGLVQPGGSLRLSCAASGFTFSSYAMSWVRQAPGKGLEWVSVIYSGGSSTYYADSVKGRFTISRD | 21 |
| anti-GPC3 | NSKNTLYLQMNSLRAEDTAVYYCARTSYLNHGDYWGQGTLVTVSS | |
| IgVL domain of | QSVLTQPPSVSAAPGQRVTISCSGTRSNIGSDYVSWYQHLPGTAPKLLVYGDNLRPSGIPDRFSASKSGTSATLG | 22 |
| anti-GPC3 | ITGLQTGDEADYYCGTWDYTLNGVVFGGGTKLTVLG | |
| anti-GPC3 scFv | QSVLTQPPSVSAAPGQRVTISCSGTRSNIGSDYVSWYQHLPGTAPKLLVYGDNLRPSGIPDRFSASKSGTSATLG | 23 |
| | ITGLQTGDEADYYCGTWDYTLNGVVFGGGTKLTVLGSRGGGGSGGGGSGGGGSLEMAQVQLVESGGGLVQ | |
| | PGGSLRLSCAASGFTFSSYAMSWVRQAPGKGLEWVSVIYSGGSSTYYADSVKGRFTISRDNSKNTLYLQMNSLR | |
| | AEDTAVYYCARTSYLNHGDYWGQGTLVTVSS | |
| anti-CD3 scFv | DVQLVQSGAEVKKPGASVKVSCKASGYTFTRYTMHWVRQAPGQGLEWIGYINPSRGYTNYADSVKGRFTITT | 24 |
| | DKSTSTAYMELSSLRSEDTATYYCARYYDDHYCLDYWGQGTTVTVSSGEGTSTGSGGSGGSGGADDIVLTQSP | |
| | ATLSLSPGERATLSCRASQSVSYMNWYQQKPGKAPKRWIYDTSKVASGVPARFSGSGSGTDYSLTINSLEAEDA | |
| | ATYYCQQWSSNPLTFGGGTKVEIK | |
| BsAb linker | TSGGGGS | 25 |
| (Linker 2) | | |
| GPC3/CD3 BsAb | QSVLTQPPSVSAAPGQRVTISCSGTRSNIGSDYVSWYQHLPGTAPKLLVYGDNLRPSGIPDRFSASKSGTSATLG | 26 |
| | ITGLQTGDEADYYCGTWDYTLNGVVFGGGTKLTVLGSRGGGGSGGGGSGGGGSLEMAQVQLVESGGGLVQ | |
| | PGGSLRLSCAASGFTFSSYAMSWVRQAPGKGLEWVSVIYSGGSSTYYADSVKGRFTISRDNSKNTLYLQMNSLR | |
| | AEDTAVYYCARTSYLNHGDYWGQGTLVTVSSTSGGGGSDVQLVQSGAEVKKPGASVKVSCKASGYTFTRYTM | |
| | HWVRQAPGQGLEWIGYINPSRGYTNYADSVKGRFTITTDKSTSTAYMELSSLRSEDTATYYCARYYDDHYCLDY | |
| | WGQGTTVTVSSGEGTSTGSGGSGGSGGADDIVLTQSPATLSLSPGERATLSCRASQSVSYMNWYQQKPGKAP | |
| | KRWIYDTSKVASGVPARFSGSGSGTDYSLTINSLEAEDAATYYCQQWSSNPLTFGGGTKVEIK | |
| scFv linker | SRGGGGSGGGGSGGGGSLEMA | 27 |
| (Linker 1) | | |
| Anti- | EVQLVQSGAEVKKPGESLTISCKASGYSFPNYWITWVRQMSGGGLEWMGRIDPGDSYTTYNPSFQGHVTISID | 28 |
| AFP158/HLA | KSTNTAYLHWNSLKASDTAMYYCARYYVSLVDIWGQGTLVTVSS | |
| heavy chain | | |
| variable domain, | | |
| protein | | |
| Anti- | QSVLTQPASVSGSPGQSITISCTGTSSDVGGYNYVSWYQQHPGKAPKLMIYDVNNRPSEVSNRFSGSKSGNTA | 29 |
| AFP158/HLA | SLTISGLQAEDEADYYCSSYTTGSRAVFGGGTKLTVLG | |
| light chain | | |
| variable domain, | | |
| protein | | |
| Signal peptide | SETDTLLLWVLLLWVPGSTG | 30 |
| Anti AFP scFv | QSVLTQPASVSGSPGQSITISCTGTSSDVGGYNYVSWYQQHPGKAPKLMIYDVNNRPSEVSNRFSGSKSGNTA | 31 |
| | SLTISGLQAEDEADYYCSSYTTGSRAVFGGGTKLTVLGSRGGGGSGGGGSGGGGSLEMAEVQLVQSGAEVKKP | |
| | GESLTISCKASGYSFPNYWITWVRQMSGGGLEWMGRIDPGDSYTTYNPSFQGHVTISIDKSTNTAYLHWNSLK | |
| | ASDTAMYYCARYYVSLVDIWGQGTLVTVSS | |
| Linker | | 32 |
| CD28 ECD partial | IEVMYPPPYLDNEKSNGTIIHVKGKHLCPSPLFPGPSKP | 33 |
| CD28 TMD | FWVLVVVGGVLACYSLLVTVAFIIFWV | 34 |
| CD28 ICD | RSKRSRLLHSDYMNMTPRRPGPTRKHYQPYAPPRDFAAYRS | 35 |
| CD3ζ ICD | RVKFSRSADAPAYQQGQNQLYNELNLGRREEYDVLDKRRGRDPEMGGKPRRKNPQEGLYNELQKDKMAEAY | 36 |
| | SEIGMKGERRRGKGHDGLYQGLSTATKDTYDALHMQALPPR | |
| Anti-PSMA HC | QVQLVQSGAEVKKPGASVKLSCKASGYTFTYFDINWVRQTPEQGLEWMGGISPGDGNTNYNENFKGRVTM | 37 |
| variable | TRDTSSSTAYMELSRLRSDDTAVYYCARDGNFPYYAMDSWGQGTTVTVSS | |
| Linker 1 | GGGGSGGGGSGGGGS | 38 |
| Anti-PSMA LC | DVVMTQSPLSLPVTLGEPASISCRSSQSLVYSNGNTYLHWYQQKPGQSPRLLIYKVSNRFSGVPDRFSGSGSGT | 39 |
| variable | DFTLKISRVEAEDVGVYFCSQSTHVPYTFGQGTKLEIKS | |
| Linker 2 | GGGGS | 40 |
| Anti-CD3 HC | EVQLVESGGGLVQPGGSLKLSCAASGFTFNKYAMNWVRQAPGKGLEWVARIRSKYNNYATYYADSVKDRFTI | 41 |
| variable | SRDDSKNTAYLQMNNLKTEDTAVYYCVRHGNFGNSYISYWAYWGQGTLVTVSS | |
| Linker 3 | GGGGSGGGGSGGGGS | 42 |
| Anti-CD3 LC | QTVVTQEPSLTVSPGGTVTLTCGSSTGAVTSGNYPNWVQQKPGQAPRGLIGGTKFLAPGTPARFSGSLLGGKA | 43 |
| variable | ALTLSGVQPEDEAEYYCVLWYSNRWVFGGGTKLTVL | |
| Anti-PSMA HC | YFDIN | 44 |
| CDR1 | | |
| Anti-PSMA HC | GDGNTNYNENFK | 45 |
| CDR2 | | |
| Anti-PSMA HC | DGNFPYYAMDS | 46 |
| CDR3 | | |
| Anti-PSMA LC | RSSQSLVYSNGNTYLH | 47 |
| CDR1 | | |
| Anti-PSMA LC | KVSNRFS | 48 |
| CDR2 | | |
| Anti-PSMA LC | SQSTHVPYT | 49 |
| CDR3 | | |
| Anti-CD3 HC | KYAMN | 50 |
| CDR1 | | |
| Anti-CD3 HC | RIRSKYNNYATYYADSVKD | 51 |
| CDR2 | | |
| Anti-CD3 HC | HGNFGNSYISYWAY | 52 |
| CDR3 | | |
| Anti-CD3 LC | GSSTGAVTSGNYPN | 53 |
| CDR1 | | |
| Anti-CD3 LC | GTKFLAP | 54 |
| CDR2 | | |
| Anti-CD3 LC | VLWYSNRWV | 55 |
| CDR3 | | |
| CD28 co- | RSKRSRLLHSDYMNMTPRRPGPTRKHYQPYAPPRDFAAYRS | 56 |
| stimulatory | | |
| fragment 2 | | |
| 4-1BB co- | KRGRKKLLYIFKQPFMRPVQTTQEEDGCSCRFPEEEEGGCEL | 57 |
| stimulatory | | |
| fragment 2 | | |
| OX40 co- | ALYLLRRDQRLPPDAHKPPGGGSFRTPIQEEQADAHSTLAKI | 58 |
| stimualtory | | |
| fragment 2 | | |
| anti-CD47 scFv | DIVMTQTPLSLPVTPGEPASISCRSSQSLVHSNGNTYLHWYQQKPGKAPKLLIYKVSYRFSGVPDRFSGSGSGTD | 59 |
| | FTLKISRVEAEDVGVYYCSQNTHVPRTFGQGTKVEIKRSRGGGGSGGGGSGGGGSLEQVQLQESGPGLVKPSQ | |
| | TLSLTCTVSGYTFTNYYVFWVRQARGQRLEWIGDINPVNGDTNFNEKFKNRVTISADKSISTAYLQWSSLKASD | |
| | TAMYYCARGGYTMDYWGQGTLVTVSS | |
| IgVH domain of | QVQLQESGPGLVKPSQTLSLTCTVSGYTFTNYYVFWVRQARGQRLEWIGDINPVNGDTNFNEKFKNRVTISAD | 60 |
| anti-CD47 | KSISTAYLQWSSLKASDTAMYYCARGGYTMDYWGQGTLVTVSS | |
| IgVL domain of | DIVMTQTPLSLPVTPGEPASISCRSSQSLVHSNGNTYLHWYQQKPGKAPKLLIYKVSYRFSGVPDRFSGSGSGTD | 61 |
| anti-CD47 | FTLKISRVEAEDVGVYYCSQNTHVPRTFGQGTKVEIKR | |
| IgVH domain of | EVQLVQSGAEVKKPGESLKISCKGSGYSFTSYWIGWVRQMPGKGLEWMGIIYPGDSDTRYSPSFQGQVTISAD | 62 |
| anti-CD19 | KSISTAYLQWSSLKASDTAMYYCARQVWGWQGGMYPRSNWWYNLDSWGQGTLVTVSS | |
| antibody | | |
| IgVL domain of | LPVLTQPPSVSVAPGKTARITCGGNNIGSKSVHWYQQKPGQAPVLVVYDDSDRPSGIPERFSGSNSGNTATLTI | 63 |
| anti-CD19 | SRVEAGDEADYYCQVWDSSSDYVVFGGGTKLTVLG | |
| antibody | | |
| IgVH domain of | QVQLVQSGAEVKKPGESLKVSCKASGYTFTSYYMHWVRQAPGQGLEWMGIINPSGG | 64 |
| anti-CD16A | STSYAQKFQGRVTMTRDTSTSTVYMELSSLRSEDTAVYYCARGSAYYYDFADYWGQ GTLVTVSS | |
| IgVL domain of | SYVLTQPSSVSVAPGQTATISCGGHNIGSKNVHWYQQRPGQSPVLVIYQDNKRPSGIP | 65 |
| anti-CD16A | ERFSGSNSGNTATLTISGTQAMDEADYYCQVWDNYSVLFGGGTKLTVL | |
| PSMA HC | QVQLVQSGAEVKKPGASVKLSCKASGYTFTYFDINWVRQTPEQGLEWMGGISPGDGNTNYNENFKGRVTM | 66 |
| variable linker | TRDTSSSTAYMELSRLRSDDTAVYYCARDGNFPYYAMDSWGQGTTVTVSS GGGGSGGGGSGGGGS | |
| LC variable | DVVMTQSPLSLPVTLGEPASISCRSSQSLVYSNGNTYLHWYQQKPGQSPRLLIYKVSNRFSGVPDRFSGSGSGT | |
| | DFTLKISRVEAEDVGVYFCSQSTHVPYTFGQGTKLEIKS | |
| AFP peptide | FMNKFIYEI | 67 |
| aa158-166 | | |
| Full length GPC3 | MAGTVRTACLVVAMLLSLDFPGQAQPPPPPPDATCHQVRSFFQRLQPGLKWVPETPVPGSDLQVCLPKGPTC | 68 |
| | CSRKMEEKYQLTARLNMEQLLQSASMELKFLIIQNAAVFQEAFEIVVRHAKNYTNAMFKNNYPSLTPQAFEFV | |
| | GEFFTDVSLYILGSDINVDDMVNELFDSLFPVIYTQLMNPGLPDSALDINECLRGARRDLKVFGNFPKLIMTQVS | |
| | KSLQVTRIFLQALNLGIEVINTTDHLKFSKDCGRMLTRMWYCSYCQGLMMVKPCGGYCNVVMQGCMAGVV | |
| | EIDKYWREYILSLEELVNGMYRIYDMENVLLGLFSTIHDSIQYVQKNAGKLTTTIGKLCAHSQQRQYRSAYYPEDL | |
| | FIDKKVLKVAHVEHEETLSSRRRELIQKLKSFISFYSALPGYICSHSPVAENDTLCWNGQELVERYSQKAARNGMK | |
| | NQFNLHELKMKGPEPVVSQIIDKLKHINQLLRTMSMPKGRVLDKNLDEEGFESGDCGDDEDECIGGSGDGMIK | |
| | VKNQLRFLAELAYDLDVDDAPGNSQQATPKDNEISTFHNLGNVHSPLKLLTSMAISVVCFFFLVH | |
| PSA peptide | KLQCVDLHV | 69 |
| Anti-AFP CAR | ATGGAAACCGATACACTGCTGCTGTGGGTGCTGCTGCTGTGGGTGCCAGGATCCACAGGCCAGAGCGTGC | 70 |
| DNA | TGACACAGCCTGCCTCCGTGTCTGGCTCTCCTGGCCAGTCCATCACCATCAGCTGTACCGGCACCAGCTCCG | |
| | ACGTGGGCGGCTACAATTACGTGTCCTGGTATCAGCAGCATCCCGGCAAGGCCCCCAAGCTGATGATCTAC | |
| | GACGTGAACAACCGGCCCAGCGAGGTGTCCAACAGATTCAGCGGCAGCAAGAGCGGCAACACCGCCAGC | |
| | CTGACAATCAGCGGACTGCAGGCCGAGGACGAGGCCGACTACTACTGCAGCAGCTACACCACCGGCAGC | |
| | AGAGCCGTGTTTGGCGGAGGCACCAAGCTGACAGTGCTGGGCAGTAGAGGCGGCGGAGGAAGCGGAGG | |
| | CGGAGGATCTGGCGGAGGGGGATCTCTGGAAATGGCCGAGGTGCAGCTGGTGCAGTCTGGCGCCGAAG | |
| | TGAAGAAGCCTGGCGAGAGCCTGACCATCTCCTGCAAGGCCAGCGGCTACAGCTTCCCCAACTACTGGAT | |
| | CACCTGGGTGCGCCAGATGTCCGGCGGAGGCCTGGAATGGATGGGCAGAATCGACCCCGGCGACAGCTA | |
| | CACAACCTACAACCCCAGCTTCCAGGGCCACGTGACCATCAGCATCGACAAGAGCACCAATACCGCCTACC | |
| | TGCACTGGAACAGCCTGAAGGCCTCCGACACCGCCATGTACTACTGCGCCCGGTACTATGTGTCCCTGGTG | |
| | GATATCTGGGGCCAGGGCACACTCGTGACCGTGTCTAGCGCCGCTGCAATTGAAGTGATGTACCCCCCTCC | |
| | CTACCTGGACAACGAGAAGTCCAACGGCACCATCATCCACGTGAAGGGCAAGCACCTGTGCCCCAGCCCT | |
| | CTGTTTCCTGGGCCCTCCAAGCCTTTCTGGGTGCTGGTGGTCGTGGGCGGAGTGCTGGCCTGTTACTCCCT | |
| | GCTCGTGACAGTGGCCTTCATCATCTTTTGGGTGCGCAGCAAGCGGAGCAGACTGCTGCACAGCGACTAC | |
| | ATGAACATGACCCCCAGACGGCCAGGCCCCACCAGAAAGCACTACCAGCCTTACGCCCCTCCCAGAGACTT | |
| | CGCCGCCTACCGGTCCAGAGTGAAGTTCAGCAGAAGCGCCGACGCCCCTGCCTACCAGCAGGGACAGAAC | |
| | CAGCTGTACAACGAGCTGAACCTGGGCAGACGGGAAGAGTACGACGTGCTGGACAAGCGGAGAGGCAG | |
| | GGACCCTGAGATGGGCGGCAAGCCCAGAAGAAAGAACCCCCAGGAAGGCCTGTATAACGAACTGCAGAA | |
| | AGACAAGATGGCCGAGGCCTACAGCGAGATCGGCATGAAGGGCGAGCGGAGAAGAGGCAAGGGCCACG | |
| | ATGGCCTGTACCAGGGCCTGAGCACCGCCACAAAGGACACCTACGACGCACTGCACATGCAGGCCCTGCC | |
| | CCCCAGA | |
| Signal Peptide | ATGGAAACCGATACACTGCTGCTGTGGGTGCTGCTGCTGTGGGTGCCAGGATCCACAGGC | 71 |
| DNA | | |
| Anti-CAR scFv | CAGAGCGTGCTGACACAGCCTGCCTCCGTGTCTGGCTCTCCTGGCCAGTCCATCACCATCAGCTGTACCGG | 72 |
| DNA | CACCAGCTCCGACGTGGGCGGCTACAATTACGTGTCCTGGTATCAGCAGCATCCCGGCAAGGCCCCCAAG | |
| | CTGATGATCTACGACGTGAACAACCGGCCCAGCGAGGTGTCCAACAGATTCAGCGGCAGCAAGAGCGGC | |
| | AACACCGCCAGCCTGACAATCAGCGGACTGCAGGCCGAGGACGAGGCCGACTACTACTGCAGCAGCTACA | |
| | CCACCGGCAGCAGAGCCGTGTTTGGCGGAGGCACCAAGCTGACAGTGCTGGGCAGTAGAGGCGGCGGA | |
| | GGAAGCGGAGGCGGAGGATCTGGCGGAGGGGGATCTCTGGAAATGGCCGAGGTGCAGCTGGTGCAGTC | |
| | TGGCGCCGAAGTGAAGAAGCCTGGCGAGAGCCTGACCATCTCCTGCAAGGCCAGCGGCTACAGCTTCCCC | |
| | AACTACTGGATCACCTGGGTGCGCCAGATGTCCGGCGGAGGCCTGGAATGGATGGGCAGAATCGACCCC | |
| | GGCGACAGCTACACAACCTACAACCCCAGCTTCCAGGGCCACGTGACCATCAGCATCGACAAGAGCACCA | |
| | ATACCGCCTACCTGCACTGGAACAGCCTGAAGGCCTCCGACACCGCCATGTACTACTGCGCCCGGTACTAT | |
| | GTGTCCCTGGTGGATATCTGGGGCCAGGGCACACTCGTGACCGTGTCTAGC | |
| CD28 partial ECD | ATTGAAGTGATGTACCCCCCTCCCTACCTGGACAACGAGAAGTCCAACGGCACCATCATCCACGTGAAGGG | 73 |
| DNA | CAAGCACCTGTGCCCCAGCCCTCTGTTTCCTGGGCCCTCCAAGCCT | |
| CD28 TMD DNA | TTCTGGGTGCTGGTGGTCGTGGGCGGAGTGCTGGCCTGTTACTCCCTGCTCGTGACAGTGGCCTTCATCAT | 74 |
| | CTTTTGGGTG | |
| CD28 ICD DNA | CGCAGCAAGCGGAGCAGACTGCTGCACAGCGACTACATGAACATGACCCCCAGACGGCCAGGCCCCACCA | 75 |
| | GAAAGCACTACCAGCCTTACGCCCCTCCCAGAGACTTCGCCGCCTACCGGTCC | |
| CD3ζ ICD DNA | AGAGTGAAGTTCAGCAGAAGCGCCGACGCCCCTGCCTACCAGCAGGGACAGAACCAGCTGTACAACGAG | 76 |
| | CTGAACCTGGGCAGACGGGAAGAGTACGACGTGCTGGACAAGCGGAGAGGCAGGGACCCTGAGATGGG | |
| | CGGCAAGCCCAGAAGAAAGAACCCCCAGGAAGGCCTGTATAACGAACTGCAGAAAGACAAGATGGCCGA | |
| | GGCCTACAGCGAGATCGGCATGAAGGGCGAGCGGAGAAGAGGCAAGGGCCACGATGGCCTGTACCAGG | |
| | GCCTGAGCACCGCCACAAAGGACACCTACGACGCACTGCACATGCAGGCCCTGCCCCCCAGA | |
| Anti-AFP CAR | METDTLLLWVLLLWVPGSTGQSVLTQPASVSGSPGQSITISCTGTSSDVGGYNYVSWYQQHPGKAPKLMIYDV | 77 |
| full aa sequence | NNRPSEVSNRFSGSKSGNTASLTISGLQAEDEADYYCSSYTTGSRAVFGGGTKLTVLGSRGGGGSGGGGSGGG | |
| | GSLEMAEVQLVQSGAEVKKPGESLTISCKASGYSFPNYWITWVRQMSGGGLEWMGRIDPGDSYTTYNPSFQ | |
| | GHVTISIDKSTNTAYLHWNSLKASDTAMYYCARYYVSLVDIWGQGTLVTVSSAAAIEVMYPPPYLDNEKSNGTII | |
| | HVKGKHLCPSPLFPGPSKPFWVLVVVGGVLACYSLLVTVAFIIFWVRSKRSRLLHSDYMNMTPRRPGPTRKHYQ | |
| | PYAPPRDFAAYRSRVKFSRSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| Anti-PSMA-Anti- | QVQLVQSGAEVKKPGASVKLSCKASGYTFTYFDINWVRQTPEQGLEWMGGISPGDGNTNY | 78 |
| CD3 BsAb full | NENFKGRVTMTRDTSSSTAYMELSRLRSDDTAVYYCARDGNFPYYAMDSWGQGTTVTVSS | |
| sequence | GGGGSGGGGSGGGGSDVVMTQSPLSLPVTLGEPASISCRSSQSLVYSNGNTYLHWYQQKP | |
| | GQSPRLLIYKVSNRFSGVPDRFSGSGSGTDFTLKISRVEAEDVGVYFCSQSTHVPYTFGQ | |
| | GTKLEIKSGGGGSEVQLVESGGGLVQPGGSLKLSCAASGFTFNKYAMNWVRQAPGKGLEW | |
| | VARIRSKYNNYATYYADSVKDRFTISRDDSKNTAYLQMNNLKTEDTAVYYCVRHGNFGNS | |
| | YISYWAYWGQGTLVTVSSGGGGSGGGGSGGGGSQTVVTQEPSLTVSPGGTVTLTCGSSTG | |
| | AVTSGNYPNWVQQKPGQAPRGLIGGTKFLAPGTPARFSGSLLGGKAALTLSGVQPEDEAE | |
| | YYCVLWYSNRWVFGGGTKLTVL | |
| Anti-AFP HC | GYSFPNYW | 79 |
| CDR1 | | |
| Anti-AFP HC | IDPGDSYT | 80 |
| CDR2 | | |
| Anti-AFP HC | ARYYVSLVDI | 81 |
| CDR3 | | |
| Anti-AFP LC | SSDVGGYNY | 82 |
| CDR1 | | |
| Anti-AFP LC | DVN | 83 |
| CDR2 | | |
| Anti-AFP LC | SSYTTGSRAV | 84 |
| CDR3 | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (e.g., in the claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A cell comprising:
a. a first nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and
b. a second nucleic acid comprising a second nucleotide sequence encoding an antigen-binding protein which binds to a second target and a third target, wherein the second nucleotide sequence is operably linked to an inducible promoter, wherein expression of the second nucleotide sequence is activated upon binding of the first target to the cell surface receptor.

2. The cell of claim 1, wherein the first nucleotide sequence is operably linked to a constitutive promoter.

3. The cell of claim 2, wherein the constitutive promoter is a human promoter.

4. The cell of claim 3, wherein the human promoter is human elongation factor-1 alpha (EF-1α) or a functional variant thereof.

5. The cell of claim 4, wherein the human promoter comprises a nucleotide sequence of SEQ ID NO: 1.

6. The cell of any one of the previous claims, wherein the inducible promoter comprises a binding site for a protein expressed or activated upon binding of the first target to the cell surface receptor.

7. The cell of claim 6, wherein the protein is a transcription factor expressed in activated T-cells.

8. The cell of claim 7, wherein the transcription factor is an Nuclear Factor of Activated T-Cells (NFAT), AP-1, or NF-κB.

9. The cell of claim 7 or 8, wherein the transcription factor is IRF4, Rel, T-bet, Blimp-1, BATF, or Nur77.

10. The cell of any one of the previous claims, comprising a third nucleic acid comprising a third nucleotide sequence encoding a protein which is exogenous to the cell, wherein the third nucleotide sequence is operably linked to an inducible promoter comprising a binding site for a transcription factor expressed in activated T-cells, wherein the expression of the exogenous protein activates expression of the second nucleotide sequence.

11. The cell of any one of the previous claims, wherein the first target is an antigen expressed by a tumor or cancer cell or an antigen of a pathogen.

12. The cell of any one of the previous claims, wherein the second target is expressed on the surface of an immune cell.

13. The cell of any one of the previous claims, wherein the third target is an antigen expressed by a tumor or cancer cell or an antigen of a pathogen.

14. The cell of any one of the previous claims, wherein each of the first target and the third target is an antigen expressed by a tumor or cancer cell.

15. The cell of any one of the previous claims, wherein the first target is different from the third target.

16. The cell of claim 15, wherein the first target is expressed by the same type of cell expressing the third target.

17. The cell of any one of claims 14 to 16, wherein the tumor or cancer cell is a cell of a solid tumor.

18. The cell of claim 17, wherein the first target and/or third target is present on the surface of the cells of the solid tumor.

19. The cell of any one of the previous claims, wherein the first target and/or third target is a peptide bound to an MHC molecule.

20. The cell of any one of the previous claims, wherein the first target is an alpha fetoprotein (AFP) peptide bound to an MHC molecule.

21. The cell of claim 20, wherein the MHC molecule is an MHC Class I molecule.

22. The cell of claim 21, wherein the MHC Class I molecule is a human MHC Class I molecule.

23. The cell of claim 22, wherein the human MHC Class I molecule is an HLA-A*02 molecule.

24. The cell of any one of claims 12-23, wherein the immune cell is a T-cell.

25. The cell of claim 24, wherein second target is CD3.

26. The cell of any one of claims 12-23, wherein the immune cell is a natural killer (NK) cell.

27. The cell of claim 26, wherein second target is CD16a.

28. The cell of any one of the previous claims, wherein the third target is selected from the group consisting of: GPC3, CD47, MUC16, CD19, CD20, CD22, EpCAM, EGFR, HER2 CEA, PSMA, AFP, PSA, BCMA, FCRL5, NY-ESO, HPV16, and FoxP3.

29. The cell of claim 28, wherein the third target is GPC3.

30. The cell of any one of the previous claims, wherein the ECD of the cell surface receptor comprises an antibody protein product which binds to the first target.

31. The cell of claim 30, wherein the antibody protein product is a single-chain Fragment variable (scFv) or a tandem scFv.

32. The cell of claim 31, wherein the scFv comprises one, two, or three heavy chain CDR sequences of an anti-AFP scFV.

33. The cell of claim 32, wherein the scFv comprises one, two, or three heavy chain CDR sequences of SEQ ID NOs: 79-81.

34. The cell of claim 33, wherein the scFV comprises a heavy chain variable region sequence of SEQ ID NO: 28.

35. The cell of claim 31, wherein the scFv comprises one, two, or three light chain CDR sequences of an anti-AFP scFV.

36. The cell of claim 32, wherein the scFv comprises one, two, or three light chain CDR sequences of SEQ ID NOs: 82-84.

37. The cell of claim 36, wherein the scFV comprises a light chain variable region sequence of SEQ ID NO: 29.

38. The cell of any one or claims 32-37, wherein the anti-AFP scFv comprises an amino acid sequence of SEQ ID NO: 31.

39. The cell of any one of claims 1-38, wherein the ECD of the cell surface receptor comprises a ligand-binding domain of a cell surface receptor exogenous to the cell.

40. The cell of claim 39, wherein the ECD comprises a ligand-binding domain of SIRP1a or EGFR.

41. The cell of any one of claims 1-38, wherein the ECD of the cell surface receptor comprises a receptor-binding domain of a ligand.

42. The cell of claim 41, wherein the ECD comprises a receptor-binding domain of CD47 or EGF.

43. The cell of any one of the previous claims, wherein the TMD of the cell surface receptor comprises at least a portion of a T-cell signaling molecule.

44. The cell of claim 43, wherein the TMD of the cell surface receptor comprises at least a portion of a T-cell signaling molecule which is different from the T-cell signaling molecule of the ICD.

45. The cell of claim 43, wherein the TMD of the cell surface receptor comprises at least a portion of a T-cell signaling molecule which is the same as the T-cell signaling molecule of the ICD.

46. The cell of any one of the previous claims, wherein the TMD of the cell surface receptor does not comprise a transmembrane domain of a T-cell receptor.

47. The cell of any one of claims 43-46, wherein the TMD comprises at least a portion of CD28, 4-1BB, or OX40.

48. The cell of any one of the previous claims, wherein the ICD of the cell surface receptor comprises at least a portion of CD3, CD28, 4-1BB, or OX40.

49. The cell of claim 48, wherein the ICD comprises a cytoplasmic portion of CD3-ζ.

50. The cell of any one of the previous claims, wherein the ICD of the cell surface receptor comprises at least a portion of two different T-cell signaling molecules.

51. The cell of claim 50, wherein the ICD comprises the cytoplasmic portion of each of the two T-cell signaling molecules.

52. The cell of claim 50 or 51, wherein each of the two T-cell signaling molecules of the ICD is independently selected from the group consisting of: CD3, CD28, 4-1BB, and OX40.

53. The cell of claim 52, wherein the ICD comprises the cytoplasmic portion of CD3-ζ and the cytoplasmic portion of CD28.

54. The cell of any one of the previous claims, wherein the cell surface receptor further comprises additional portions of a T-cell signaling molecule.

55. The cell of claim 54, wherein the cell surface receptor further comprises a portion of the ECD of CD28.

56. The cell of any one of the previous claims, wherein the cell surface receptor comprises one or more amino acid sequences of SEQ ID NOs: 28-36 and 79-84.

57. The cell of any one of the previous claims, wherein the antigen-binding protein is a bispecific antibody.

58. The cell of any one of claims 1 to 56, wherein the antigen-binding protein is a multispecific antibody.

59. The cell of any one of the previous claims, comprising a vector comprising both the first nucleic acid and the second nucleic acid.

60. The cell of any one of claims 1 to 58, comprising a first vector and a second vector, wherein the first vector comprises the first nucleic acid and the second vector comprises the second nucleic acid.

61. The cell of any one of the previous claims, which is an immune cell.

62. The cell of any one of the previous claims, which is a T lymphocyte or a natural killer (NK) cell.

63. The cell of claim 62, wherein the T lymphocyte is CD8+, CD4+, CD8+/CD4+, or a T-regulatory cell.

64. The cell of any one of the previous claims, which is genetically engineered to silence the expression of an endogenous TCR.

65. A composition comprising (i) a nucleic acid comprising a first nucleotide sequence encoding a cell surface receptor comprising an extracellular domain (ECD) which binds to a first target, a transmembrane domain (TMD), and an intracellular domain (ICD) comprising at least a portion of a T-cell signaling molecule, and (ii) a nucleic acid comprising a second nucleotide sequence encoding an antibody which binds to a second target and a third target, wherein the second nucleotide sequence is operably linked to an inducible promoter, wherein expression of the second nucleotide sequence is activated upon binding of the first target to the cell surface receptor.

66. The composition of claim 65, comprising the first nucleotide sequence operably linked to a constitutive promoter.

67. The composition of claim 66, wherein the constitutive promoter is a human promoter.

68. The composition of claim 67, wherein the human promoter is human elongation factor-1 alpha (EF-1α) or a functional variant thereof.

69. The composition of claim 68, wherein the human promoter comprises a nucleotide sequence of SEQ ID NO: 1.

70. The composition of any one of claims 65-69, wherein the inducible promoter comprises a binding site for a protein expressed upon binding of the first target to the cell surface receptor.

71. The composition of claim 70, wherein the protein is a transcription factor expressed in activated T-cells.

72. The composition of claim 71, wherein the transcription factor is an Nuclear Factor of Activated T-Cells (NFAT), AP-1, or NF-κB.

73. The composition of claim 71, wherein the transcription factor is IRF4, Rel, T-bet, Blimp-1, BATF, or Nur77.

74. The composition of any one of claims 65-73, wherein the nucleic acid comprising the first nucleotide sequence also comprises the second nucleotide sequence.

75. The composition of claim 74, comprising a vector comprising both the first nucleotide sequence and the second nucleotide sequence.

76. The composition of any one of claims 65 to 75, wherein the nucleic acid comprising the first nucleotide sequence is distinct from the nucleic acid comprising the second nucleotide sequence.

77. The composition of claim 76, comprising a first vector and a second vector, wherein the first vector comprises the first nucleotide sequence and the second vector comprises the second nucleotide sequence.

78. A method of making a therapeutic cell, comprising contacting a cell with the composition any one of claims 65-77.

79. The method of claim 78, wherein the cell is an immune cell.

80. The method of claim 78 or 79, wherein the cell is obtained from a human.

81. The method of claim 80, comprising obtaining immune cells from a human then contacting the cells with the expression vector system.

82. The method of claim 81, comprising culturing the cells for a time period sufficient to expand the cells to a population of at least 10⁶ cells.

83. A pharmaceutical composition comprising the cells of any one of claims 1-56, the composition of any one of claims 65-77, the therapeutic cells produced by the method of any one of claims 78-82, or a combination thereof, and a pharmaceutically acceptable carrier, diluent, or excipient.

84. An article of manufacture comprising the cells of any one of claims 1-56, the composition of any one of claims 65-77, the therapeutic cells produced by the method of any one of claims 78-82, or a combination thereof, housed in a container.

85. A kit comprising the cells of any one of claims 1-64, the composition of any one of claims 65-77, the therapeutic cells produced by the method of any one of claims 78-82, or a combination thereof, and a device for administration of the cells, composition, or therapeutic cells.

86. A method of killing a diseased or infected cell, comprising contacting the diseased or infected cell with the cells of any one of claims 1-64, the composition of any one of claims 65-77, the therapeutic cells produced by the method of any one of claims 78-82, or a combination thereof.

87. The method of claim 86, wherein the contacting is carried out in vivo.

88. The method of claim 86, wherein the contacting is carried out in vitro.

89. A method of treating a subject with a disease, comprising administering to the subject the pharmaceutical composition of claim 83 in an amount effective to treat the disease, wherein the first target and third target are expressed by a cell of the disease.

90. The method of claim 89, wherein the disease is a cancer optionally selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer and thyroid cancer.

91. The method of claim 90, wherein the disease is an infection by a pathogen.

92. The method of claim 91, wherein the pathogen is a virus.

93. A method of treating a viral infection in a subject, comprising administering to the subject the pharmaceutical composition of claim 83 in an amount effective to treat the viral infection, wherein the first target and/or third target are viral antigens.

94. The method of claim 93, wherein the viral infection is caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), Human immunodeficiency virus (HIV), and Hepatitis C Virus (HCV).

95. The method of any one of claims 86-94, wherein the cells are autologous to the subject.

96. A method of treating a subject with a tumor or cancer or a viral infection, comprising (a) obtaining immune cells from a subject, (b) contacting the cells with a composition of any one of claims 65-77, and (c) administering the cells to the subject in an amount effective to treat the tumor or cancer or viral infection.

97. An immune cell comprising:
a. a nucleic acid encoding a chimeric antigen receptor (CAR) which binds to a first antigen; and
b. a nucleic acid encoding an antibody which binds to a second antigen and third antigen,
wherein the first antigen is different from the third antigen,
wherein the expression of the nucleic acid encoding a bispecific antibody is activated upon binding of the first antigen to the cell surface receptor,
wherein the first antigen and/or third antigen is a peptide of an intracellular protein bound to an MHC molecule.

98. A T-cell comprising:
a. a first nucleic acid comprising a first nucleotide sequence encoding a chimeric antigen receptor (CAR) comprising an extracellular domain (ECD) which binds to a first target, a transmembrane domain (TMD), and an intracellular domain (ICD) comprising at least a portion of a T-cell signaling molecule, and
b. a second nucleic acid comprising a second nucleotide sequence encoding a bispecific antibody which binds to a second target and a third target, wherein the second nucleotide sequence is operably linked to an inducible promoter, wherein expression of the second nucleotide sequence is activated upon binding of the first target to the CAR,
wherein each of the first target and third target is an antigen expressed by a solid tumor, wherein the first target is different from the third target, and
wherein the second target is expressed on the surface of a T-cell or a natural killer cell.

99. A T-cell comprising:
a. a first nucleic acid comprising a first nucleotide sequence encoding a chimeric antigen receptor (CAR) comprising an extracellular domain (ECD) which binds to an alpha fetoprotein (AFP) peptide bound to an MHC molecule, a transmembrane domain (TMD), and an intracellular domain (ICD) comprising at least a portion of CD28 and CD3, and
b. a second nucleic acid comprising a second nucleotide sequence encoding a bispecific antibody which binds to GPC3 and CD3, wherein the second nucleotide sequence is operably linked to an inducible NFAT promoter, wherein expression of the second nucleotide sequence is activated upon binding of the AFP peptide bound to the MHC molecule to the CAR.
